(19) United States
(12) Patent Application Publication
(10) Pub. No.: US 2026/0210693 A1
(43) Pub. Date: Jul. 23, 2026

(54) APPARATUS FOR OPTICAL COHERENCE TOMOGRAPHY

(71) Applicant: Siloton Limited, Nottingham (GB)

(72) Inventors: Alasdair Benjamin Price, Bristol (GB); Euan John Allen, Bristol (GB); Jake Biele, Bristol (GB)

(73) Assignee: Siloton Limited, Nottingham (GB)

(21) Appl. No.: 19/132,497

(22) PCT Filed: Nov. 20, 2023
(86) PCT No.: PCT/GB2023/053032

§ 371 Date: May 23, 2025

(30) Foreign Application Priority Data
Nov. 23, 2022 (GB) 2217524.4

(51) Int. Cl. G01B 9/02091; G01B 9/02

(52) U.S. Cl. CPC G01B 9/02091; G01B 9/02051; G01B 9/02054

(57) ABSTRACT

There is presented an apparatus for an OCT system for measuring a first target sample and a second target sample. The apparatus comprises an optical arrangement configured to receive light from a light source and split the received light to propagate along at least a reference optical path and a probe optical path. The apparatus can receive light back from the reference optical path and the probe optical path and interfere said light. A light switching arrangement is configured to: receive light from the probe optical path and controllably switch the received light between at least: a) a first optical path for directing light towards the first target sample; b) a second optical path for directing light towards the second target sample.

### CROSS REFERENCE TO RELATED APPLICATION(S)

This application claims priority to International Patent Application No. PCT/GB2023/053032, filed Nov. 20, 2023, and United Kingdom Patent Application Serial No. 2217524.4, filed Nov. 23, 2022, the entire disclosures both of which are hereby incorporated by reference

### TECHNICAL FIELD

The field of the present invention is apparatus for Optical Coherence Tomography (OCT), in particular, but not exclusively, apparatus for ocular OCT.

### BACKGROUND

The general operation of an OCT system is now described. OCT systems are used to scan the depth of a sample. An interferometer configuration is used wherein input light from a light source is split by an optical splitter into a reference path and a probe path. The probe path leads its light to a sample for testing. The sample may have different features within its structure that reflect some or all of the light. These features may be at different depths. For example, if the OCT system is an ocular OCT system, then the sample is an eye. The eye's retina has layers that a medical professional may wish to investigate. The light wavelength chosen is one that can penetrate, but also be reflected by, the different layers. The wavelength is also selected to ensure it can penetrate the aqueous humor and so reach the retina in the first place. Light reflecting off a top layer, nearest to the incoming probe light may be captured by the OCT system and travel back towards an interfering element such as a combining beam splitter. The optical splitter and combining beam splitter are often the same component. Furthermore, light reflecting off a deeper retina layer, further away from the incoming probe light than the top layer, may also be captured by the OCT system and travel back towards the same interfering element. The light reflecting off the top layer travels a shorter distance between the optical splitter and interfering element than the light reflected off the deeper layer.

The light travelling down the reference path is typically incident upon a moveable reflecting element that diverts the reference light towards the same interfering element that receives the reflected probe light. The reflecting element is typically a mirror that is moveable by being mounted on a translation stage. The mirror is moveable such that at different mirror positions, the reference light travels different distances between the optical splitter and interfering element. The OCT is typically set-up such that the total optical path length from optical splitter to interfering element for probe light reflecting off the top retina layer is equal, or near equal to, to the total optical path length from optical splitter to interfering element for reference light reflecting off the mirror in a first position (call this a ‘near position’). Equally, the total optical path length from optical splitter to interfering element for probe light reflecting off the deeper retina layer is equal to, or near equal to, the total optical path length from optical splitter to interfering element for reference light reflecting off the mirror in a second position (call this a ‘far position’). The mirror is moveable between its near and far positions and typically other positions that correspond to the optical path taken by probe light reflecting off other retina layers. Near and far positions may also vary from sample to sample (eye to eye) and so scan range of delay can be wider than the abovesaid ‘near-far’ range for any one sample, but includes all near-far ranges for all samples to be measured. The different positions are often referred to as ‘z-position’ and correspond to the depth scanning of the eye or position of the imaging window the OCT system is able to resolve. When probe light and reference light travel the same distance or similar distance (within the light's coherence length) between optical splitter and interfering element, they interfere at the interfering element. This interference is detected by the OCT system and signifies a reflection from that depth portion of the retina. Typically, a broadband or wavelength-swept light source is used such that a full depth profile (a-scan) can be reconstructed without the requirement to scan the axial z position. Various detection techniques and mechanisms exist including, for example using a spectrometer or balanced detectors.

An OCT system, such as an ocular OCT system, typically operates by focusing the broadband probe light onto a spot on the surface of the retina wall and maintaining the light at that spot whilst the mirror is z-scanned about some or all of its translation range such that the axial imaging range covers the region of the sample that is to be imaged. Probe light enters the retina and is reflected in different proportions from layers of the structure and then returns to the OCT system. The light is spectrally resolved and then measured. After some post-processing, including but not limited to a Fourier transform of the spectral data, a one-dimension depth profile of the sample can be attained providing details of the structure. Some OCT systems also move the spot to another part of the retina wall and then do further depth scans in either one dimension to produce a “b-scan”, or two dimensions to produce a “c-scan” or “enface” three-dimensional image.

The above general operation of an OCT system is not just used for eyes but other biological samples or non-biological samples.

Most existing OCT systems, in particular ocular OCT systems, measure one eye at a time (hence are monocular in design) wherein a patient has their first eye measured, then either the patient moves their head, or the OCT system is moved, to get the second eye measured. This can be inconvenient for the patient and may take excess time to measure both eyes.

U.S. Pat. No. 9,888,841B2 describes a system and method for obtaining ophthalmic measurements whereby a device is configured to be head mountable, automatically axially length aligned with a selected target, and laterally aligned so that light from an OCT source enters through the pupil of the eye under test. The frame of the head mountable OCT is customizable, capable of analysing both the left and right eye of a subject. The device can be operated by the person undergoing test. Embodiments include mechanisms for eye fixation, lateral, angular and depth scanning of target regions. FIG. 2 of this document depicts a head mountable OCT device with the frame and an OCT photonic module attached to the frame such that the probe beam of the OCT photonic module enters the target first eye. In FIG. 4 of this document, the OCT photonic module is attached to the frame in a further manner that aligns its probe beam with the second eye of the individual as the target eye. Therefore, the OCT photonic module tests each eye by being attachable to and detachable from a frame in at least two configurations, such that in a first configuration the OCT photonic module is aligned with a first eye and in a second configuration is aligned with the second eye. The reconfiguration of this apparatus to test each eye is time consuming and inconvenient.

US20210025691A1 describes a wearable electronic device and an optical arrangement for optical coherence tomography. The optical arrangement comprises at least one lens configured to shape a beam of light from the optical coherence tomography device and at least one mirror positioned so that light from the lens is incident on the at least one mirror and wherein the at least one mirror is configured to move in at least one direction relative to the optical coherence tomography device. FIGS. 1 and 2 of this document only show a set-up configured to interrogate a single sample. Such an apparatus is not optimally configured to measure two eyes. FIG. 6 shows apparatus being worn on the persons torso and does not show head mounted apparatus.

US20210085184A1 describes an apparatus, an optical coherence tomography device, a wearable electronic device and method of forming an apparatus for optical coherence tomography. The apparatus comprises at least one layer of silicon dioxide and an integrated optoelectronic circuit. The integrated optoelectronic circuit comprises an interferometer configured for optical coherence tomography wherein the integrated optoelectronic circuit is formed within a layer of silicon dioxide. This document shows the use of an integrated opto-electronic circuit for use in OCT but does not address binocular systems. FIG. 7 shows apparatus being worn on the person's torso and does not show head mounted apparatus. WO2009/120544 describes an optical coherence tomography instrument that comprises an eyepiece for receiving at least one eye of a user; a light source that outputs light that is directed through the eyepiece into the user's eye; an interferometer configured to produce optical interference using light reflected from the user's eye; an optical detector disposed so as to detect said optical interference; and electronics coupled to the detector.

### SUMMARY

There is presented an apparatus for an Optical Coherence Tomography, OCT, system for measuring a first target sample and a second target sample; the apparatus comprising: I) an optical arrangement configured to: i) receive light from a light source; ii) split the received light to propagate along at least: a reference optical path; and, a probe optical path; iii) receive light back from the reference optical path; iv) receive light back from the probe optical path; v) interfere the light received back from the reference and probe optical paths; II) a light switching arrangement configured to:
receive light from the probe optical path, controllably switch the received light between at least: a) a first optical path for directing light towards the first target sample; b) a second optical path for directing light towards the second target sample.

The apparatus may also: vi) detect the interfered light. The apparatus may comprise a light-detector to receive the interfered light. The apparatus may comprise a detecting arrangement comprising a wavelength demultiplexer for receiving the interfered light and outputting at least: a) a first portion of the light towards one or more detectors; b) a second portion of the light towards the one or more detectors, wherein the first portion of light comprises a different wavelength than the second portion of light. The wavelength demultiplexer may be configured to output any number of different wavelength channels towards the one or more detectors including any of, but not limited to: two or more channels; three or more channels; five or more channels; ten or more channels; fifteen or more channels; twenty or more channels. The detector may be a CCD device and/or a line scanner. The apparatus may be configured to output all the wavelength channels to the same detector.

The apparatus described above may be adapted in any way with configurations and/or features described herein, including, but not limited to any one or more of the following options and any one or more of the options of the first aspect listed below.

The first sample and second sample areas may be for the same sample object or a different sample object. The sample may be a biological tissue sample. The biological tissue sample may form part of a dead or living biological organism or may have been extracted from a living organism. Other samples may include non-biological samples or biological non-tissue samples.

In a first aspect there is provided an apparatus for an ocular Optical Coherence Tomography, OCT, system for measuring a first eye and a second eye; the apparatus comprising: I) an optical arrangement configured to: i) receive light from a light source; ii) split the received light to propagate along at least: a reference optical path; and, a probe optical path; iii) receive light back from the reference optical path; iv) receive light back from the probe optical path v) interfere the light received back from the reference and probe optical paths; II) a light switching arrangement configured to: receive light from the probe optical path, controllably switch the received light between at least: a) a first optical path for directing light towards the first eye; b) a second optical path for directing light towards the second eye. The apparatus may also: vi) detect the interfered light. The apparatus may be portable. The apparatus may be a wearable. The apparatus may be head mountable. The apparatus may be a binocular headset.

The apparatus of the first aspect may be adapted in any way with configurations and/or features described herein, including, but not limited to, any one or more of the following options. Reference to the ‘eye’ in the first aspect and any of its optional adaptions may equally refer to a ‘sample’ that is not an eye.

The apparatus may comprise a light-detector to receive the interfered light. The apparatus may comprise a detecting arrangement comprising a wavelength demultiplexer for receiving the interfered light and outputting at least: a) a first portion of the light towards one or more detectors; b) a second portion of the light towards the one or more detectors, wherein the first portion of light comprises a different wavelength than the second portion of light. The wavelength demultiplexer may be configured to output any number of different wavelength channels towards the one or more detectors including any of, but not limited to: two or more channels; three or more channels; five or more channels; ten or more channels; fifteen or more channels; twenty or more channels. The detector may be a CCD device and/or a line scanner. The apparatus may be configured to output all the wavelength channels to the same detector.

The light propagating along the probe optical path towards the eye (or otherwise another target sample) may be referred to as ‘probe light’ herein. Light propagating back long the probe optical path (usually in an opposite direction to the probe light) may be referred to as ‘reflected probe light’. The light switching apparatus may be configured to switch substantially all the probe light from the first optical to the second optical path and vice versa. The light switching apparatus may rely on different physical parameters in its operation, including but not limited to spatial co-ordinates, spatial vectors, wavelength, frequency, time, and/or polarisation.

Item II) of the first aspect may be adapted such that the switch does not always require receiving probe light from the probe optical path. Instead, the light switching arrangement may be located in another part of the OCT system, for example in a different location in the optical circuit. A different location may be used to optimise switching between the first eye and the second eye when using modalities including, but not limited to, wavelength switching or frequency switching. The light switching arrangement might be in a different part of the optical circuit but still allows switching between the light in the two eye paths. For example, two different wavelength bandwidths could be transmitted to each eye and then wavelength switched before an AWG (in a detector arrangement).

The apparatus may be configured such that: I) the apparatus comprises an interferometer; II) the light switching apparatus is configured to: direct light reflected back from the first eye along the first optical path to the interferometer; direct light reflected back from the second eye along the second optical path to the interferometer.

The apparatus may be configured such that: the apparatus comprises a set of one or more reflecting elements for scanning the probe light across the first eye and the second eye; the light switching apparatus configured to receive the probe light from at least one of the reflecting elements and direct the probe light towards the first eye and/or second eye.

The apparatus may be configured such that the light switching apparatus comprises a moveable mirror. The mirror may be moveable by translating along at least one axis. The mirror may be moveable by rotating about at least one axis.

The movement of the mirror may comprise moving the mirror into at least two different spatial positions. The different spatial positions may be via a rotation of the mirror or translation of the entire mirror along an axis. The different mirror positions may receive the probe path light output from an optical splitter and cause the light to propagate to at least one of the eyes. The different mirror positions may direct the light towards different eyes. A first mirror position may direct light towards the first eye whilst a second mirror position, different to the first mirror position, may direct the light towards the second eye. A rotation of the mirror into different positions may involve rotating the mirror into different orientations. The different mirror positions may optionally be configured such that, in a first position, outgoing probe light is received by the mirror and directed, by the mirror towards the first eye; in a second position the outgoing probe light is not received by the mirror (i.e., the mirror is moved out of the path of the outgoing probe light) and propagates towards the second eye. In some examples the mirror used for switching light between the eyes is the same mirror as a mirror used to scan the probe light across the eye(s). In some examples, the mirror used for switching light between eyes is a different mirror as a mirror used to scan the probe light across the eye(s). The switching mirror may receive light from a further scanning mirror. The switching mirror may divert light into at least one further mirror for scanning probe light across the eye(s). The term ‘mirror’ used above and elsewhere herein, may apply in principle to any light reflecting element.

The apparatus may be configured such that: the apparatus comprises a set of one or more reflecting elements for scanning the probe light across the first eye and the second eye; the light switching apparatus is configured to receive the probe light and direct the probe light towards the first eye and/or second eye via at least one of the reflecting elements.

The reflecting element may be configured to use the same mirror movements for scanning each eye. The reflecting element may be configured to use a first set of movements (or positions after being moved) for scanning the first eye and second set of, different, movements for scanning second eye. Multiple scanning mirrors can be used.

The apparatus may be configured such that at least one of the reflecting elements is configured to direct probe light along: a) the first optical path towards the first eye; and, b) the second optical path towards the second eye. The same reflecting mirror may be used to divert outgoing probe light into both eyes.

The apparatus may be configured such that at least one reflecting element receives: a) probe light for the first eye at a first portion of the reflecting element; b) probe light for the second eye at a second portion of the reflecting element; the first portion being a different portion than the second portion.

The apparatus may comprise one or more piezo-electric actuators (or other micro-electrical actuators) for scanning light across at least one of the eyes. The apparatus may be configured such that: a) a first piezo-electric actuator receives outgoing probe light for a first eye from a first port of a switch; and outputs the said received light towards the first eye; b) a second piezo-electric actuator receives outgoing probe light for a second eye from a second port of a switch; and outputs the said received light towards the eye. Advantages of a configuration of direct actuator-to-eye, compared to options such as MEMS or additional reflective components may be: lower loss and lower noise.

The apparatus may comprise a piezo-electric actuator for directing light either towards the first eye or the second eye. The piezo-electric actuator may be rotatable to: a) direct light to the first eye in a first orientation; b) direct light to the second eye in a second orientation. One or more further scanning mirrors may receive light from the piezo electric actuator and output light towards an eye.

The apparatus may be configured such that the interferometer is an integrated optic interferometer.

The apparatus may comprise: a) a polarisation rotator configured to receive outgoing probe light and controllably output the received light in at least a first polarisation and second polarisation; each polarisation being an output mode of the rotator; the rotator operative to be in one mode at any one time; b) a polarisation splitting element configured to allow, or direct, the probe light: i) to the first eye when in the first polarisation; ii) to the second eye when in the second polarisation.

The apparatus may be configured such that the reference optical path comprises an integrated optic waveguide reference arm extending from the interferometer towards a reference arm reflecting element; the reference arm reflecting element being integrated with the integrated optic waveguide reference arm. Integration may be a monolithic integration or a hybrid integration.

The apparatus may be configured such that the light switching arrangement comprises an integrated optic light switch. The integrated optic light switch may comprise a tunable Mach-Zehnder interferometer. The tuning may be via a heating element inducing a thermo-optic effect in at least one arm of the said Mach-Zehnder interferometer.

There is presented in a second aspect a method of operating an apparatus for an Optical Coherence Tomography, OCT, system for measuring a first target sample and a second target sample; the apparatus comprising: I) an optical arrangement configured to: i) receive light from a light source; ii) split the received light to propagate along at least: a reference optical path; and, a probe optical path; iii) receive light back from the reference optical path; iv) receive light back from the probe optical path; v) interfere the light received back from the reference and probe optical paths; II) a light switching arrangement configured to: receive light from the probe optical path; the method comprising: controllably switching the received light between at least: a) a first optical path for directing light towards the first target sample; b) a second optical path for directing light towards the second target sample. The apparatus may also: vi) detect the interfered light.

The method of the second aspect may be adapted in any way with configurations and/or features described herein, including, but not limited to, any one or more of the options for the first aspect.

In a third aspect there is presented a portable apparatus for accommodating an ocular Optical Coherence Tomography, OCT, system, the OCT system comprising: I) a set of one or more optical components for directing light from a light source to at least one eye of a user when wearing the portable apparatus; II) a set of one or more further components wherein at least one of the further components is an electrical component for controlling at least one of: i) the optical components, and/or, ii) another of the further components; the portable apparatus comprising: A) a first end face proximal to the user when using the portable apparatus and comprising at least one aperture for directing the light to the eye; B) a plurality of compartments comprising at least: a) a first compartment for accommodating the set of one or more optical components; b) a second compartment for accommodating the set of one or more further components; the second compartment spaced from the first end face by at least the first compartment; C) a wall positioned between the first compartment and second compartment and extending substantially across the portable apparatus so as to provide thermal insulation between the first compartment and the second compartment.

The portable apparatus of the third aspect may be used, for example, to accommodate at least part of the apparatus of the first aspect and any of its optional features or adaptions. The portable apparatus of the third aspect may be adapted in any way with configurations and/or features described herein, including, but not limited to any one or more of the following options.

The wall may divide at least one compartment from at least another compartment. The wall may at least partially define the extent of at least one compartment. At least one compartment may be box-like, preferably at least one of the first and second compartments is box-like. The box-like shape may comprise a set of opposite sides that are substantially flat. These sides may be parallel or close to parallel to the end face. The wall may comprise the said side of a compartment or the side of two adjacent compartments. Other sides may be substantially flat or not substantially flat, for example curved sidewalls. A side of a box-like compartment may comprise one or more through-holes for allowing objects or light to pass through and extend between adjacent compartments. Objects may include but are not limited to: optical fibres, electrical wires. In addition, or in the alternative, objects (such as but not limited to electrical and/or optical components) may be at least partially housed within the wall. The portable apparatus may be configured such that the wall is removable from the portable apparatus.

The portable apparatus may be configured such that the set of one or more optical components is mounted on the wall.

The portable apparatus may be configured such that the set of one or more further components is mounted on the wall.

The portable apparatus may comprise a housing body comprising at least: the first compartment, the second compartment; the wall; the first end face; wherein: the wall is a first wall disposed inside the housing body; the set of one or more further components being a second set of components; the first end face is an outer face of a second wall; the housing body comprises a third wall at a distal end of the housing body directly away from the user's face when in use; the third wall comprising: i) an outer face facing away from the user's face when in use; and ii); an inner face facing towards the user's face when in use; the portable apparatus comprising a third set of components mounted upon the inner face of the third wall. The portable apparatus may further comprise at least one of: i) a heat sink; ii) a fan; iii) an induction charging unit; integrated with the third wall or mounted on the outer face of the third wall.

### BRIEF DESCRIPTION OF THE DRAWINGS

Embodiments of the present invention will now be described in detail with reference to the accompanying drawings, in which:

FIG. 1 is a schematic diagram of an example of an apparatus for an OCT system;

FIG. 2 is a schematic diagram of an example of an apparatus for an ocular OCT system;

FIG. 3 is an example of an apparatus for an ocular OCT system;

FIG. 4 is an example of a detection arrangement for an OCT system;

FIGS. 5, 5a, 6, 6a, 7, 8, 9, 10, 11, 12, 13, 14 show alternative examples of switchable probe paths using the chip of FIG. 3;

FIGS. 15a and 15b show an example of a housing for accommodating an ocular OCT apparatus;

FIG. 16 shows an example of a cross section of an apparatus for accommodating an ocular OCT apparatus;

FIG. 17 shows an example of a cross section of an apparatus for accommodating an ocular OCT apparatus;

FIG. 18 shows an example of a cross section of an apparatus for accommodating an ocular OCT apparatus;

FIG. 19 shows an example of a perspective view of an apparatus for accommodating an ocular OCT apparatus;

FIGS. 20a and 20b show perspective and side views of an example of a plate-like wall for mounting components of an OCT apparatus.

### DETAILED DESCRIPTION

There is presented an apparatus for an Optical Coherence Tomography, OCT, system. The general operation of the OCT system described herein is similar to that described in the ‘general operation’ in the above background section aside. The use of the apparatus may be for clinical fields such as, but not limited to: dermatology, cardiology, urology, dentistry, audiology, gastroenterology, oncology, pulmonology, ophthalmology. Non-clinical applications of the apparatus may include any of, but not limited to: 3D imaging techniques that can be used to evaluate and inspect material surfaces, plant matter, multilayer polymer films, fibre coils, and coatings. Other applications may include: non-destructive testing, plant/leaf diagnostics, fruit/vegetable analysis, thin film/battery analysis, chemical and manufacturing process monitoring, microfluidics, art restoration, authentication/security.

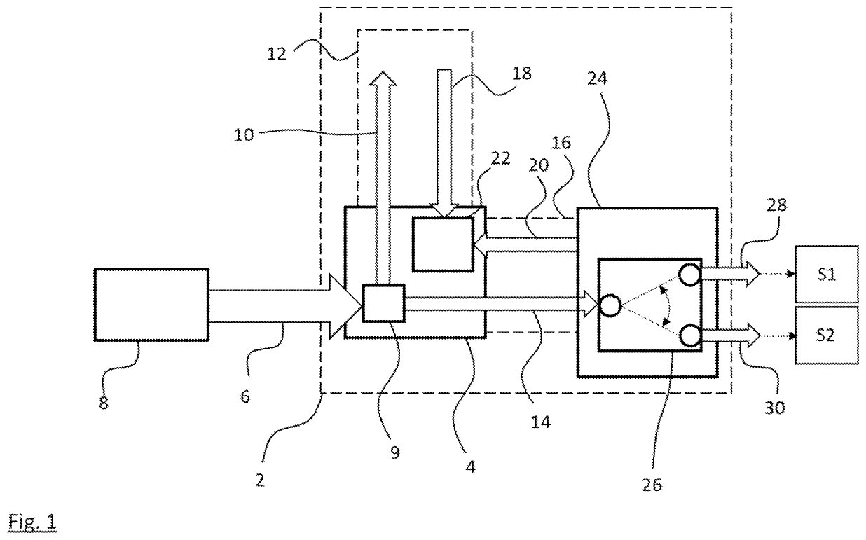

A schematic example of the apparatus 2 is shown in FIG. 1. The apparatus 2 may be used for measuring a first target sample S1 and a second target sample S2. The apparatus 2 comprises an optical arrangement 4. The optical arrangement 4 configured to receive light 6 from a light source 8. The optical arrangement 4 is further configured to split 9 the received light such that a first portion 10 of the light propagates along at least a reference optical path 12 and a second portion 14 of the light propagates along a probe optical path 16.

The optical arrangement 4 is further configured to receive light 18 back from the reference optical path 12 and receive light 20 back from the probe optical path 16. The optical arrangement 4 is further configured to interfere 22 the light 18, 20, received back from the reference 12 and probe 16 optical paths.

The apparatus 2 may comprise a detection arrangement (not shown) for detecting the interfered light. Detection apparatus may include one or more electromagnetic wave detectors including but not limited to CCD arrays, line scanning cameras, balanced photodiodes, and one or more spectrometers. The detector arrangement may comprise other components such as a wavelength demultiplexer. An example of a detection arrangement is presented elsewhere herein.

The apparatus 2 further comprises a light switching arrangement 24 configured to receive light 14 from the probe optical path 16 and controllably switch 26 the received light between at least: a) a first optical path 28 for directing light towards the first target sample S1; b) a second optical path 30 for directing light towards the second target sample S2. Although a first and second target sample are described above, the apparatus 2 may be configured to direct light to three or more target samples; for example, the light switching arrangement may be configured to controllably switch the received light between five different optical paths.

Unlike other OCT systems, the present apparatus allows for a common set of OCT components to be used for interrogating two, or more, target samples. This in turn makes the apparatus versatile and requiring less components that constructing multiple separate OCT systems. In addition, scanning each sample in a sequential fashion or in series allows for an improved signal-to-noise ratio given a maximum source power output and per-sample integration time than when the samples are imaged in parallel.

Optical components in the apparatus 2 may be adjusted for use with different users, for example, changing a position of a collimating or focusing mirror or lens to correctly adapt the optical system to a particular eye of a user or particular set of eyes of different users. For example, one or more lenses or mirror for delivering probe light for one of the user's eyes may be configured differently to the one or more lenses or mirror for delivering probe light for the other of the user's eyes. Once the optical components for the different eyes are set-up correctly, then the apparatus 2 may be used again at another time without any further user-related calibrations. This saves time and energy resources in calibration.

The apparatus may be housed in a portable apparatus such as a headset. The headset may be head-mountable and/or hand-held (akin to holding a set of binoculars). The headset may be kept by a user to perform multiple scans at different times. Furthermore, a consistent scanning configuration, allows for more consistent scans over time. In some examples, the OCT apparatus may be in a patient device that performs each eye scan per overall OCT measurement. Other, existing, OCT systems require the user to flip the device over to scan the other eye. This may entail that the device has to be adjusted for each eye, which in turn means that the device needs to be adjusted every time the patient scans. This will likely lead to variations in scans, even of the same eye.

The first sample and second sample areas may be for the same sample object or a different sample object. The sample may be a biological tissue sample. The biological tissue sample may form part of a dead or living biological organism or may have been extracted from a living organism. Other samples may include non-biological samples, or biological non-tissue samples. The apparatus 2 may be used for non-biological tissue samples, or biological non-tissue samples.

In one example, the apparatus is for an ocular Optical Coherence Tomography, OCT, system wherein the first target sample S1 is a first eye and the second target sample S2 is a second eye that is different to the first eye. The first and second eyes are typically human eyes but can be other animal eyes. The first and second eyes are typically of the same entity, i.e., the eyes of the same biological organism, but can in principle be eyes of different organisms. In the following discussions and examples presented herein, reference made to first eye and second eye may in principle apply to any target sample.

The light 14 propagating along the probe optical path 16 towards an eye (or otherwise another target sample) may be referred to as ‘probe light’ herein. Light 20 propagating back along the probe optical path 16 (usually in an opposite direction to the probe light) may be referred to as ‘reflected probe light’. The light switching arrangement 26 (also referred to as light switching apparatus) may be configured to switch substantially all the probe light from the first optical path 28 to the second optical path 30 and vice versa. The switching of the light by light switching arrangement 26 may be controlled by one or more control signals input to the light switching arrangement 26. The control signal may be generated by one or more electronic or opto-electronic control apparatus including any of, but not limited to: one or more controllers embodied in hardware and/or software, one or more computers comprising a processor and a memory wherein the memory comprises instructions which when executed by the processor act to control the light switching arrangement 26. The same or other similar control apparatus may be used to control any other feature of the apparatus and/or other associated equipment such as, but not limited to: the light source 8; any parts of the optical arrangement 4 (e.g., phase shifters used to control the splitting ratio of the splitter 9); any detection apparatus (not shown in the figure) for receiving the interfered 22 light; or any other component described in this example or any other example herein.

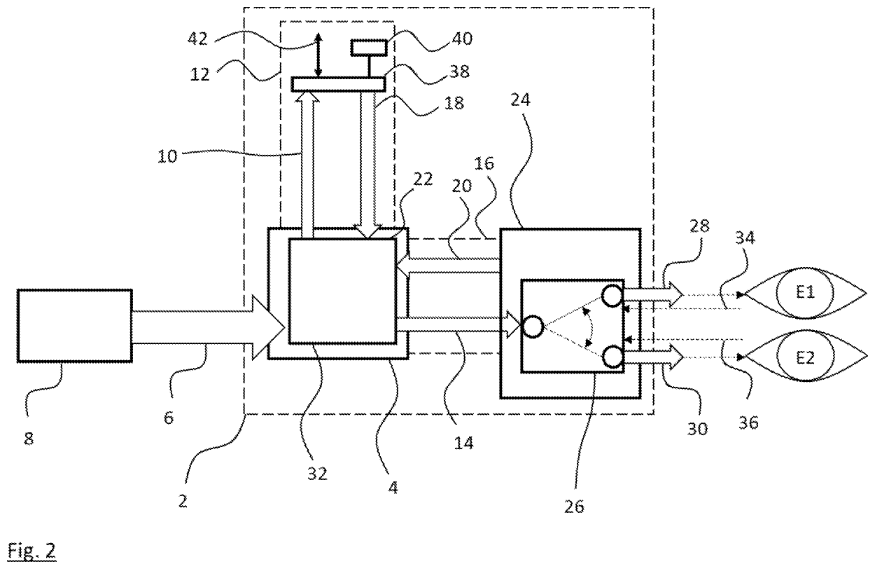

In general, for both the examples in FIG. 1, FIG. 2 underneath, and other examples herein, light may be transferred between different components using any suitable optical linking means. The means may include, but are not limited to, any one or more of: connection via free space and optionally bulk-optic components; optical fibre, integrated optics. Any of the components may be integrated with another one or more components using monolithic or hybrid integration. Hybrid integration may include utilising flip-chip, end on chip (butt) coupling or any other integration techniques. The integration may be chip upon chip or may be package level integration wherein a first packaged chip is integrated onto, or otherwise with, another one or more further packaged chips. Integration may include integrating optical fibres to chips or bulk optic component onto a chip. One or more common platforms may be used to integrate multiple components of the apparatus 2 onto, wherein the platform holds the components in optical alignment with each other. Connection to the platform may be accomplished in any way including solder, glue, friction fit or other means.

FIG. 2 shows a further schematic example similar to FIG. 1 wherein like reference numerals represent like features.

The apparatus 2, in this example, may comprise at least a portion of an interferometer 32. It is to be understood that the portion of the interferometer 32 in this example refers to the splitting and interfering component portions of the entire interferometer used for the OCT measurement wherein the interferometer arms are used as the reference arm (or ‘scanning arm’) and probe arm that directs light to the sample and captures reflected light from the sample. The interfering component portion may also be referred to as a ‘combiner’.

For the purposes of this example, the optical pathways of the interferometer associated with light that: a) is split 9; b) propagates down separate interferometer arms; and c) is interfered 22; includes i) the reference optical path 12 acting as one interferometer arm; ii) the probe optical path 16 acting as one interferometer arm. In other words, the interferometer 32 may be configured to receive reflected light 34 back from the first eye E1 and reflected light 36 back from the second eye E2. The said reflected light 34, 36 may, optionally, travel back along the same optical paths, towards (and optionally within) the optical arrangement 4 that the same light used to be previously incident upon the said eyes E1, E2. Thus, light output from, and thereafter received by, the interferometer 32 may travel along the same optical path to an eye E1, and respectively back from the said eye E1, E2. In the example of FIG. 2 the interferometer 32 is the same component that splits the light 6 from the light source 8 into the probe path 16 and reference path 12; however, the apparatus 2 may comprise a separate light splitter 9 to the interferometer 32.

The portion of the OCT interferometer 32 residing in the optical arrangement 4, preferably uses amplitude splitting to send the light down each arm but may be wavefront splitting and/or polarisation splitting. The type of interferometer is preferably of a Michelson interferometer configuration, however other interferometer designs may be used including a Mach-Zehnder configuration (MZI). The interferometer may comprise at least one optical splitter and at least two spatially separate reference arms wherein one reference arm includes the reference optical path 12 whilst the other arm includes the probe optical path 16. In other interferometer configurations there may be separate first and second splitters wherein: the second splitter acts as an optical combiner to interfere the two returns light beams 18, 20; one interferometer arm optically links the first splitter to the second splitter via the reference optical path 12 whilst another interferometer arm optically links the first splitter to the second splitter via the probe optical path 16.

As discussed above, the interferometer configuration of the apparatus 2 that directs interferometer-arm light towards the eyes E1, E2, may comprise optical pathways may include free space light-propagation paths and waveguide light-propagation paths. In particular, the probe arm may have at least part of the optical path in free space and/or via one of more waveguides. Waveguide paths may include optical fibres and/or integrated optic waveguides.

The components of the interferometer 32 the constitute at least part of the optical arrangement 4 and act to at least split the source light 6 into the probe 16 and reference 12 paths, and subsequently combine the same reflected light, may be any one or more of, but not limited to, the following configuration types:
I) A first optical splitter acting as the component to split 9 the source light 6 and a second, different, optical combiner used to receive both the reflected probe light 18 and the reflected reference light 20 and combine, hence interfere 22, the said received reflected light. Such splitters or combiners may be, for example a 2×2 MMI coupler, a 2×2 broadband directional coupler, or any other splitter/combiner design detailed elsewhere herein.
II) A common optical splitter/combiner that acts as both: a) the component to split 9 the source light 6 into the interferometer arms; and b) the optical combiner used to receive both the reflected probe light and the reflected reference light 20 and combine, hence interfere 22, the said received reflected light. Such a splitter/combiner may be, for example a 2×2 MMI coupler, a 2×2 broadband directional coupler, or any other splitter/combiner design detailed elsewhere herein.
III) One or more interferometers. In other words, the overall ‘OCT’ interferometer configuration that uses the probe 16 and reference 12 optical paths as interferometer arms may utilise, in this example, a further interferometer structure, such as a balanced Mach-Zehnder (BMZI), that acts as both: a) the component to split 9 the source light 6; and, b) the optical combiner used to receive both the reflected probe light 18 and the reflected reference light 20 and combine, hence interfere 22, the said received reflected light. In another configuration, similar to the one listed in example I) above, one BMZI may be used to split 9, whilst another different BMZI may be used to combine (hence interfere reference 18 and probe 20 light).

The splitters/combiners may be any n×n port optical splitter, for example a 1×2 port optical splitter or a 2×2 port optical splitter. The integrated optical splitter may comprise any of a Multimode Interference coupler (MMI), a Y-branch coupler, a directional coupler (DC), a star coupler or another type of coupler listed elsewhere herein. The splitting or coupling ratio of the coupler (or MZI) may be any ratio including any of: 50/50; 60/40; 40/60; 30/70; 70/30; 20/80; 80;20; 90/10; 10/90, or any other splitter ratio. Preferably, the coupling ratio may be imbalanced to direct more light along the reference path 12 than the probe path 16 to maximise the total optical power, and therefore signal, whilst also limiting the amount of light entering the eye E1, E2. The splitter or coupler may have a tunable splitting ratio.

For example, the OCT interferometer may comprise a single integrated optic waveguide 2×2 MMI or directional coupler that is used to split 9 the received light 6 from the light source into the probe and reference optical paths and receive reflected light from both said optical paths. Where the splitter or combiner used to split 9 or combine and interfere 22 is stated to be an MZI or BMZI, the MZI may in principle be a balanced MZI (BMZI) or an unbalanced MZI (UMZI), for example having one reference arm ~p phase imbalanced from the other arm. The MZI may be tunable such that light entering one input port of the MZI may be switched between two different output ports upon receiving a control signal. The switching control may be provided using any suitable effect including, but not limited to: thermo-optic effect by having one or more heating elements on at least one, preferably both interferometer arms; electro-optic effect, again used on one or both interferometer arms. The phase shifter should preferably at least be able to induce a p phase change at the centre wavelength of interest.

In this example of FIG. 2, the reference optical path 12 is configured to propagate the reference light 10 towards, and be incident upon, one or more optical elements 38 that allows at least a portion, preferably substantially all, or all of the reference light 10 to be diverted back towards the optical arrangement 4 as the reflected reference light 18. The one or more elements 38 are controlled by an actuator 40 that allows the element(s) 38 to controllably provide at least two different length optical paths between the splitting 9 and interfering means of the optical arrangement 4 that the reference light 10, 18 takes. This light diverting optical element 38 may reflect light by reflection, for example using one or more mirrors; and/or, otherwise divert light back towards the optical arrangement 4 via a loop or other optical path circulating light from the spitting means 9 back to the interfering means 22. One example of a looped arrangement is a waveguide such as an optical fibre or integrated-optic waveguide that guides the outgoing reference light 10 around one or more path-loops to become the incoming reference light 18. Actuators 40 to control the provision of different path lengths include, but are not limited to: translational actuators that move 42 a mirror back and forth to extend or shorten the total optical path length of the reference arm; one or more optical phase shifters (such as thermo-optic heaters on arms of MZI switches) to divert reference light 10 around different length optical paths and direct the light back towards the interfering means 22 as the returning reference light 18. The provision of different length optical paths for the reference arm of the OCT interferometric system allows the OCT system to scan at different depths of the target sample S1, S2.

The actuators(s) 40 may be able to control the one or more optical elements 38 to provide a continuous scan across a range of path lengths, for example by scanning a mirror continuously along a translation axis. Additionally, or alternatively, the apparatus 2 may be configured to select between a plurality of discrete path length options wherein the options are not continuous in that there exists a non-accessible path length subrange between each option, in other words, for example, path length 1 is 5 cm, path length 2 is 5.001 cm, however path lengths in between options 1 and 2 are not attainable by the apparatus. The example of the apparatus 2 comprising a plurality of discrete path length options may comprise a plurality of different length waveguide paths that are selected by controlling one or more optical switches that are configured to: a) direct the outgoing reference light 10 light along a particular path; and, b) collect the said directed light and input it back to the combiner of the interferometer 32. Such switches may include one or more MZI switches. Preferably the waveguides used for the different discrete reference arm paths are integrated optic waveguides. Optionally, the paths lengths of each different length options may be tunable. This tuneability may arise from having a tuning mechanism that controllably extends or reduces the optical path length of that said option. An example of such a tuning mechanism is one or more refractive index adjusting elements that act to change the refractive index of at least a portion of a waveguide. An example of such an element is a thermo-optic heater positioned to induce a thermo-optic effect on a waveguide path.

The apparatus 2 may use any combination of bulk-optic components, integrated optic components or optical fibre components to achieve the functions set-out herein. The apparatus 2 may be an integrated-optic apparatus. In particular any one or more of, but not limited to, the: light source 8, splitter 9; interfering combiner; reference path 12; optical element 38; probe path 16; light switching apparatus 24; and any other component of the apparatus 2; may use any combination of bulk-optic components, integrated optic components or optical fibre components.

The optical paths of the OCT interferometer light to and from eyes E1 may include eye-imaging optics such as one or more lenses or mirrors configured to focus light onto the portion of the eye, of interest, for example the cornea or retina. The apparatus preferably comprises a different set of eye imaging optics for each eye.

The OCT system may comprise an arrangement of one or more scanning elements for lateral scanning across the face of the sample. The term ‘lateral’ in this content means in directions substantially perpendicular to the direction of propagation of the probe light 28 incident upon the same. Scanning elements may include one or more mirrors or other light reflecting elements such as MEMS. The scanning elements may be controllable, via one or more scanning actuators receiving one or more control signals, to scan about in at least one axis or plane. In other words, a scanning element may be configured to receive incoming probe light 28 and controllably output and direct the incoming probe light 28 in a range of different output directions. The range of different directions preferably varying along a common output plane. The apparatus may guide the light output from the scanning elements to the sample such that each of the different light output directions is incident upon the sample at a different location across the face of the sample. Multiple scanning elements may be used. For example, an X-scanning mirror and a Y-scanning mirror may be used to direct the probe light 28 across corresponding X-direction and Y-directions with respect to the face of the sample S1, S2, wherein the ‘z’ direction is the direction substantially parallel to the incident probe light that corresponds to the depth of the sample S1, S2.

Light source 8 may be any light source including light sources outputting continuous wave (CW) and/or pulses. The light source may be configured to output pulses by direct modulation and/or external modulation, for example the output of the light source may be input into an external modulator, such as but not limited to an electro-optic MZI modulator or an electro-absorption modulator (EAM). The external modulator may be optically coupled to the light source 8 using any suitable means. Any of the light source 8 and optionally any external modulator, may be separate to the apparatus 2 and optically linked with an optical connection such as an optical fibre or free space propagation with any necessary bulk optic focusing/collimating. Alternatively, the light source may be integrated with other components. In one example the light source resides on a chip that is integrated onto, or otherwise with, a further chip containing other components such as, but not limited to the optical splitter 9 and optionally any reference arm path 12 components. In some examples the light source 8 may be a swept source.

The light source may be a semiconductor light source. The light source may comprise a Super-luminescent Light Emitting Diode (SLED). The SLED ay be an edge-emitting semiconductor light source using super-luminescence. The SLED may output a broadband pulse covering the wavelength range of interest. The light source may comprise a plurality of light sources. The light source may be tunable. Additionally, or alternatively the light source may comprise a plurality of fixed wavelength light sources (for example an array of distributed feedback (DFB) lasers) wherein the apparatus is configured to controllably select one or more of the sources to output at any one time.

The apparatus may comprise one or more polarisation controlling components (for example a polarisation controller) that may optionally be included within the apparatus 2 between any two or more components. The polarisation controlling component(s) may be fibre-optic based, bulk-optic-based or integrated-optic based. The polarisation controllers may be integrated with other components or separate to them and optically linked by free space light propagation and/or optical fibre(s). The light source may also include a laser, or otherwise, driven ring resonator structure that generates a broadband or frequency comb light source.

The apparatus may use any wavelength of operation required for testing the samples S1, S2. Example wavelengths include, but are not limited to: wavelength ranges in the Near IR (800-2500 nm), wavelength ranges in the visible (380-700 nm), a wavelength centred on 1310 nm (with an example bandwidth of 100 nm) for applications such as in vivo brain imaging; a wavelength centred on 1000 nm (with an example bandwidth of 120 nm) for applications such as retina imaging; a wavelength centred on 1300 nm (with an example bandwidth of 420 nm) for applications such as food-stuff imaging; a wavelength centred on 800 nm (with an example bandwidth of 170 nm) for applications such as cornea imaging; a wavelength centred on 555 nm (with an example bandwidth of 156 nm) for applications such as ex vivo brain imaging; a wavelength centred on 560 nm (with an example bandwidth of 100 nm) for applications such as retina imaging. Preferably the present apparatus 2 uses any of, but not limited to: a centre wavelength of 850 nm with example bandwidths ranging from 10-100 nm (for example 60 nm at the 3 dB bandwidth; or 30 nm at the full width half maximum (FWHM)); a centre wavelength of 1030 nm with example bandwidths ranging from 10-100 nm; a centre wavelength of 1550 nm with example bandwidths ranging from 10-100 nm.

Accordingly, the wavelength range of operation may directly affect the components used in the apparatus 2 due to transmission windows and/or other optical performance considerations. For example, if the apparatus 2 uses integrated optic components, then the integrated optics materials may include any one or more of, but not limited to: silica, polymer; silicon, silicon nitride; indium phosphide; other semiconductors or dielectrics doped; or versions of any of these options; as the cladding and/or core waveguide materials wherein the core waveguide comprises a higher refractive index than the cladding materials. The integrated optic waveguides may comprise any one or more of, but not limited to: buried channel waveguides (also known as strip waveguides); ridge waveguides, rib waveguides.

In any of the OCT systems, the apparatus may further comprise a detection arrangement configured to receive light output from the interfering element 22 (for example an optical combiner or an MZI used to perform both the splitting 9 and interfering 22) and detect the light using one or more detectors. The detectors may be photodiodes, cameras, CMOS line scanners; CCD arrays or other types of detector. The detection arrangement may optionally comprise one or more further other components such as optical components to wavelength demultiplex the output interfered light into different physically separated wavelength channels that may be incident onto different detectors or different areas of a common detector. Upon receiving the incoming light, the detectors may output corresponding electrical signals. The detection arrangement may comprise one or more post-processing element such as a computer to process the signals and determine one or more sets of data or information associated with the OCT measurement.

It is to be understood that this example in FIG. 2, and the example in FIG. 1, may be adapted with components, configurations (and/or steps where a process or method is involved) and other features as presented in other examples herein. The adaption may be by addition or replacement. Existing features of the examples in FIGS. 1 and 2, may be removed. Correspondingly, it is to be understood that other examples herein may be adapted with components, configurations (and/or steps where a process or method is involved) and other features as presented in the examples in FIGS. 1 and 2. Such features may include, but are not limited, to any one or more of: details about any of the optical paths; details about the target sample; details about the electromagnetic signals; details about the light source; details about the optical arrangement 2 including details about the interferometer, control mechanism, component structure and material, waveguides, mirrors or other reference light controlling/diverting components; light switching arrangement and details therein including switch type and control; other components such as free space/bulk optics for directing the light towards the target samples S1, S2; overall apparatus components including chips, packaging, integration techniques; operation of the apparatus, including measurement processes, light source and scanning control; any other features that are combinable between different examples herein.

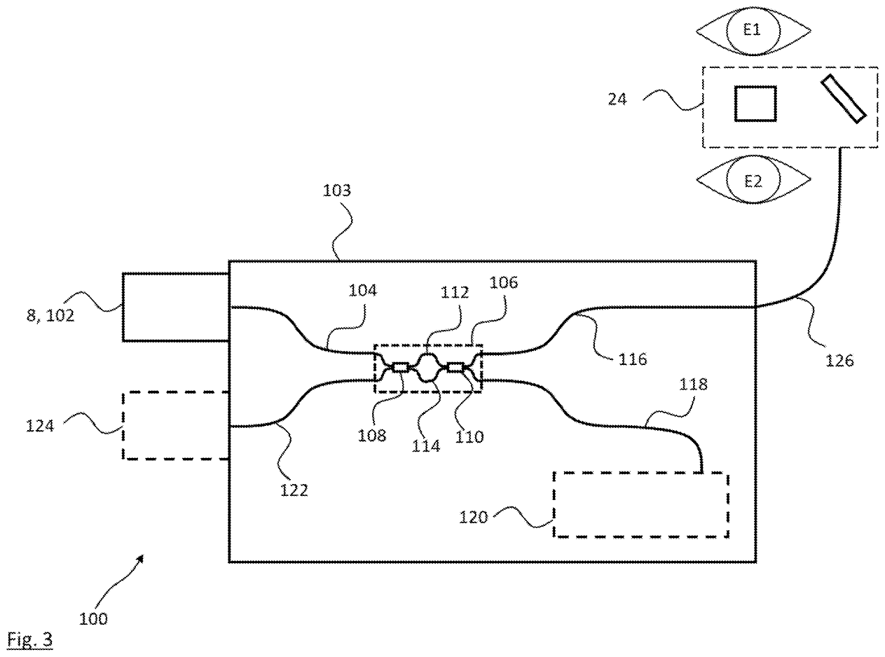

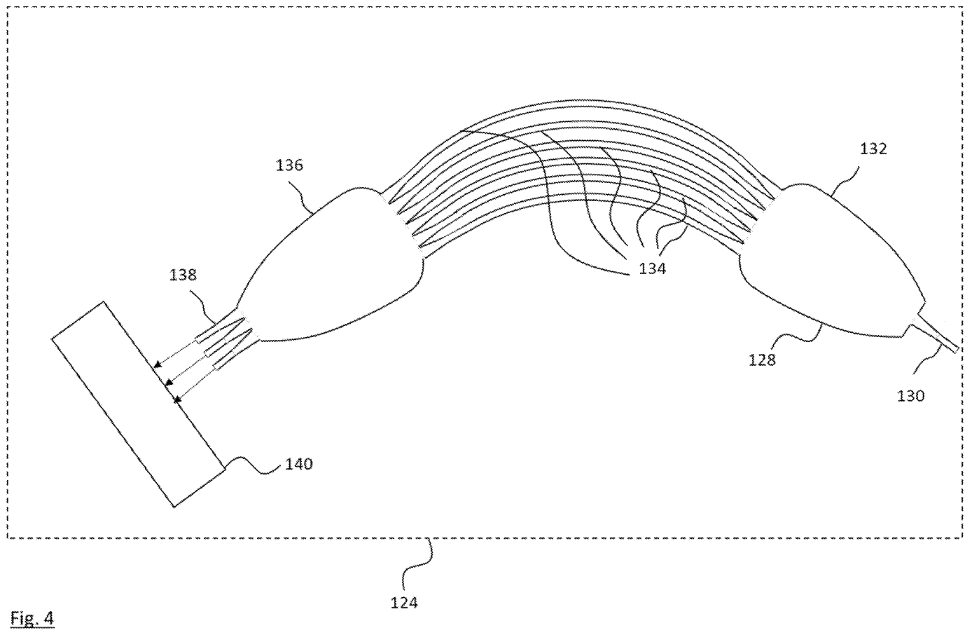

FIG. 3 shows a preferred example 100 of the apparatus 2. FIG. 4 shows a detection arrangement used for this example, and optionally other examples of the apparatus 2. It is to be understood that the example in FIG. 3, as described below, may be adapted according to any of the features described in the examples for FIGS. 1 and 2 above. Furthermore, the example in FIGS. 3 and 4 may be adapted according to other examples herein, for example the apparatus configurations given in the figures pertaining to OCT apparatus following FIG. 4.

FIG. 3 shows a chip 102 having with an integrated light source 8. The light source 8 is an SLED with a centre wavelength of 850 nm and a 60 nm bandwidth at 3 dB down from wavelength centre peak. The light source 8 inputs light into another chip 103 and is hybrid integrated with it. The light source 8 is edge coupled to the chip 103. The chip 103 comprises a first integrated optic waveguide 104 that has a terminal end at or near an end facet of the chip 103 that faces and optically aligns with the light source 8 to receive input light 6. The first waveguide 104 guides the received light into an integrated optic balanced MZI 106 with four input/output channels, each channel being an optical waveguide. The first waveguide 104, thus, is also a first one of the four input/output waveguides of the BMZI 106. The BMZI 106 comprises a first 2×2 port, 50/50 splitting ratio, MMI coupler 108 and second 2×2 port, 50/50 splitting ratio MMI coupler 110. The MMI couplers may be designed be broadband in that the splitting ratio is substantially similar over the wavelength range of interest. Other broadband couplers may be used instead of MMIs (for example broadband directional couplers). The BMZI 106 further comprises a first arm 112 and a second arm 114 wherein the first and second arms are equal in length and optical path length and optically connect the first MMI 108 to the second MMI 110. Each arm uses a different port of each of the MMI couplers 110, 108. At least one of the arms comprises a phase shifting element (not shown) such as a thermo-optic heating element for changing the phase of light propagating down at least one of the arms to induce a relative arm phase shift to controllably direct varying degrees of light between the input/output ports of the BMZI that optically link to the OCT interferometer's probe and reference arms.

The BMZI 106 comprises a second input/output waveguide 116 that forms part of the probe path and optically links the BMZI to the further components for directing light to and receiving light from the eyes E1, E2.

The BMZI 106 comprises a third input/output waveguide 118 that forms part of the reference path and optically links the BMZI to a depth scanning arrangement 120 comprising components scanning in z, as described elsewhere herein. The depth scanning arrangement is shown on chip in FIG. 3 as a schematic box, but may take any form as described elsewhere herein including being physically separate (non-integrated with) the chip 103. In this example of FIG. 3 the depth scanning arrangement 120 includes series of different path lengths that light may travel along and return back to propagate back along third waveguide 118 towards the BMZI 106. The selection of the different paths is via the operation of one or more optical switches. In this example, the selectable different path lengths and MZI switches of the z-scanning arrangement are all formed of integrated optic waveguides on the same chip 103, but other arrangements are also possible such as having the arrangement in optical fibre off-chip.

The BMZI 106 comprises a fourth input/output waveguide 122 that optically links the BMZI to the detector arrangement 124, an example of which is shown in FIG. 4. The detector arrangement 124 is shown to be edge coupled to the chip 103 and is typically hybrid integrated with the chip 103, however other configurations, mechanical connection and optical couplings are possible. Any of the detector arrangement 124 may be integrated monolithically on the chip 103, for example the wavelength demultiplexer 128 (described below) may be monolithically integrated on the chip 103 wherein each of the waveguides 138 are incident upon a chip edge for outputting different light wavelengths along different free space or fibre-optic paths. The detector 140 may also be directly integrated onto 103.

The second input/output waveguide 116 is shown to run continuously to an edge of the chip 103 wherein it optically couples to one or more further components for sending light to a set of apparatus. The one or more further components comprising the light switching arrangement 24 is schematically shown in FIG. 3. FIG. 3 shows the second input/output waveguide 116 edge coupling to an optical fibre, however other configurations may be used as shown in other figures herein, including but not limited to edge coupling to free space.

The OCT interferometer configuration in FIG. 3 therefore uses the BMZI 106 as both the light splitting 9 element and the interfering element 22 of FIGS. 1 and 2. The OCT-interferometer reference arm is the arm comprising the third waveguide 118 as well as any optical paths used in the depth scanning arrangement 120. The OCT-interferometer probe arm is the arm comprising the second waveguide 116 as well as any optical paths used in the light switching arrangement 24 and any other optical path required to transmit light to/from the eyes E1, E2 back to the BMZI 106.

FIG. 4 shows a preferred example of a detector arrangement 124. The detector arrangement 124 comprises a wavelength demultiplexer 128 for receiving input light that was output from the fourth input/output waveguide 122 and outputting different wavelength components of the received input light to spatially separate output channels that each input light into a detector. In this example of FIG. 4 the wavelength demultiplexer is an integrated optic arrayed waveguide grating (AWG) comprising an input waveguide 130 optically linking to a first star coupler 132 that splits the input light into a plurality of arrayed waveguides 134, each of different optical path length and optically linked to a second star coupler 136. The second star coupler 136 comprises a plurality of output waveguides 138, wherein the AWG is designed to output different wavelength passbands in each of the output waveguides 138.

In this example, each of the output waveguides 138 output light to be incident upon a CMOS line scanner 140 that can monitor the output light intensity of each channel. Other configurations are possible including a CCD array of a dedicated detector for each output channel 138.

In operation, with respect to FIGS. 3 and 4, the apparatus 100 outputs an SLED pulse which edge-couples into first waveguide 104 via a chip edge facet and propagates along to the first MMI 108 of the BMZI 106. The pulse is split into approximately two equal amplitude sub pulses that propagate along the BMZI arms 112, 114 and recombine at the second MMI 110. The one or more thermo-optic phase shifters that are configured to act upon at least a portion of one of the said arms 112, 114 imparts a relative phase shift between the said arms (for example below p) such that 20% of the light exits MMI 110 from the second waveguide 116 and 80% of the light exits from the third waveguide 118.

The light now travelling along the reference arm (third) waveguide 118 enters the depth scanning arrangement 120 and traverses one of the selectable optical path lengths to become incident back into the third waveguide 118 and counter propagate back down the waveguide back towards the BMZI 106.

Similarly, light travelling along second waveguide 116 becomes the probe arm light and exits the chip 103 and is directed to one of the eyes E1, E2 (the light switching apparatus 24 is set to direct the light to one of E1, E2). In this example the apparatus is configured to focus the light onto the retina. Reflected light at different tissue depths of the retina is captured by the same light delivery optical system of the apparatus (for example a set of one or more scanning mirrors and lenses) and propagates back down the optical fibre 126 and back into second waveguide 116 into the BMZI 106. Reflected portions of the probe arm light that have not traversed the same total optical path back to the BMZI are output by the BMZI into the first 104 and fourth 122 waveguides and become background noise. Reflected probe light that has travelled a total optical path length equal to (or within the coherence length of the SLED pulse) interferes constructively with the back-propagating reference path light such that at least part of the constructively interfered back propagating light enters the fourth waveguide 122 and then enters the detector arrangement 124 wherein different wavelength components of the light are separated by the AWG 128. The CMOS line scanner 140 monitors each AWG output waveguide to detect which wavelengths have more constructive interference, wherein each spot position of the line scan equates to a different wavelength. From this one run of the OCT system, the apparatus 100 may reconstruct a full axial profile (a-scan) of the sample under interrogation. The apparatus 100 may be used such that the selectable optical path of the z scanning arrangement 120 allows the best axial imaging window to be achieved for the desired purpose of the scan.

The above operation is that selected for: a) one eye E1 or E2; b) one x-scan position; c) one y-scan position; d) one z-scan depth. Any of the above variables a)-d) above may be changed and the above process run again. Preferably a method of operating the apparatus may perform the following:
I) Select an eye using the switch arrangement 24;
II) Select an x position using the appropriate scanning element;
III) Select an y position using the appropriate scanning element;
IV) Select a z-depth using the depth scanning arrangement 120;
V) Output a pulse from the SLED and conduct a measurement as discussed in the operation section above;
VI) Repeat steps III), IV) and V) for different positions of y;
VII) Repeat steps II), III), IV) V) and VI) for different positions of x;
VIII) Select the other eye of E1 and E2 and repeat steps II)-VII) again.

The raw detected optical signal may go through post processing to reconstruct the depth profile. This often includes, but is not limited to, measurement of the spectral amplitude across the source bandwidth, conversion from wavelength space to wavenumber space, amplitude renormalisation & background subtraction, and a Fourier transform converting wavenumber to real spatial information such that the data correlates reflectivity values with different depths.

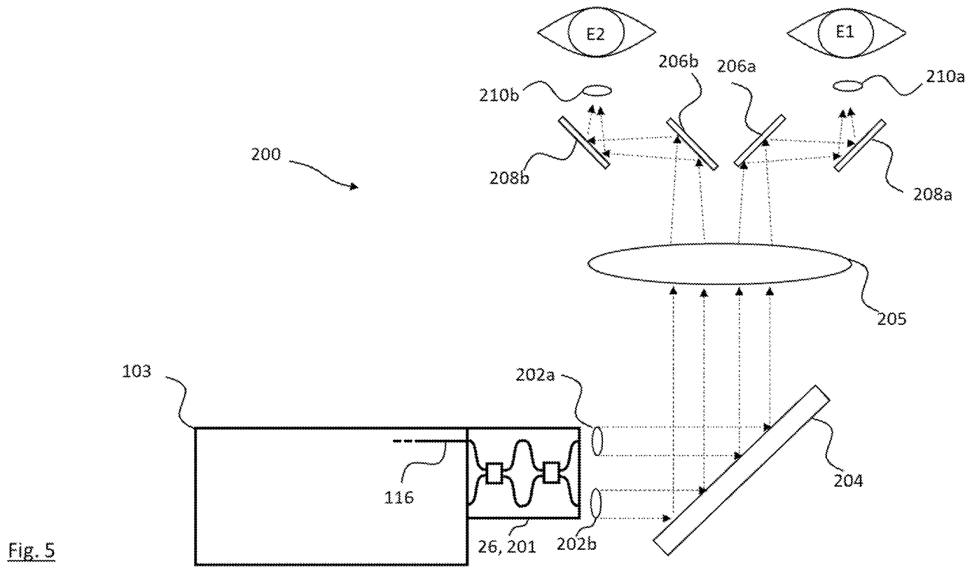

As discussed, the apparatus comprises a light switching arrangement 24. FIGS. 5-14 show different examples of light switching arrangement 24 that may be used with FIGS. 3 and 4 as well as FIGS. 1 and 2. The apparatus is described in FIGS. 5-14 primarily for ocular use but may be for any OCT use, hence for testing any target sample. For each of FIG. 5-14, it is assumed the basic components of the chip 103 are used and each example may be adapted according to any of the other examples herein, including for FIGS. 1 and 2. FIGS. 5-14 primarily show the details of different probe path configurations wherein the features of the figures are not intended to be to scale or represent any real-world dimensions. Like references in different figures represent like components.

FIG. 5 shows an example 200 using the chip 103 from FIG. 3 having probe output path 116. A further chip (or otherwise device) 201 is optically linked to chip 103 and comprises a controllable switch 26 that acts to output the probe light along one of two spatially separate output optical paths. The functionality of the further chip 201 may alternatively be monolithically or hybrid integrated onto chip 103. The switch on the chip 201 is a BMZI with arm phase shifters to control the output port probe light exits from. Other types of switch component may be used. Each BMZI output port is coupled out of the chip via respectively, two collimating lenses 202a, 202b. Each collimating lens 202a/b outputs its collimated probe light onto a common MEMS 204 (or other spatial light modulator). The light output from lens 202a is incident upon a different portion of the MEMS 204 than the light output from lens 202b. This may be that the light partially overlaps or is incident on a non-overlapping area of the MEMS 204. The MEMs is controllable to provide 2D lateral scans of the eyes E1, E2 (i.e., X and Y scanning). It is understood that any of the collimating lenses or other lenses herein, in any example may be replaced (with any corresponding component placement reconfigurations) by collimating or equivalent mirrors that provide the same light divergence or convergence.

Light reflected from MEMS 204 in incident upon a further lens 205. Light collimated from 202a uses a different region of the lens 205 than light from lens collimating lens 202b (either partially overlapping or non-overlapping regions).

For collimated light from 202a, lens 205 together with lens 210a focusses light into the eye E1 via a set of mirrors 206a, 208a. Light, for E1 is incident from lens 205 upon mirror 206a, and reflects off it to be incident upon mirror 208a to reflect off it and be incident upon lens 210a for focusing into eye E1. Reflected light from eye E1 is captured by lens 210a and travels back down the same optical path to arrive at the chip 103.

For collimated light from 202b, lens 205 together with lens 210b focusses light into the eye E2 via a set of mirrors 206b, 208b. Light, for E2 is incident from lens 205 upon mirror 206b, and reflects off it to be incident upon mirror 208b to reflect off it and be incident upon lens 210b for focusing into eye E2. Reflected light from eye E2 is captured by lens 210b and travels back down the same optical path to arrive at the chip 103.

Other lens and mirror arrangements may be possible including not requiring lens 205. This arrangement therefore uses a common MEMS 204 and a common lens 205 for scanning both eyes E1, E2.

FIG. 5a is an example similar to FIG. 5 except that the light switching MZI is a UMZI (otherwise known as an asymmetric MZI (AMZI)) as shown on chip 201a. A UMZI wavelength demultiplexes incoming light from one input port of the UMZI into: a) a first set (or comb) of wavelengths that exit a first output port of the UMZI and enter collimating lens 202a; b) a second set (or comb) of wavelengths, that are different to the first set, that exit a second output port of the UMZI and enter collimating lens 202b. The apparatus 200 in FIG. 5a may therefore transmit a first portion of the probe light to the first eye and a second portion of the probe light into the second eye simultaneously. Some implementations of the optical circuit on chip 103 may be optimised for the first set (or comb) of wavelengths that enter collimating lens 202a and, at a different time, may be optimised for the second set (or comb) of wavelengths that enter collimating lens 202b. In this case, the postprocessing software, or any other software component of the OCT system, may choose to filter out the second set of wavelengths while chip 103 is optimised for the first set of wavelengths, and may choose to filter out the first set of wavelengths while chip 103 is optimised for the second set of wavelengths. The function of the software filter or switch may instead be provided by additional hardware including, but not limited to, controllable shutters on the paths to E1 and E2, a tunable bandpass filter, a periodic bandpass filter, a set of bandpass filters, a tunable bandstop (or notch) filter, a periodic bandstop (or notch) filter, or a set of bandstop (or notch) filters. The apparatus 200 in FIG. 5a may therefore transmit a first portion of the probe light to the first eye and a second portion of the probe light into the second eye sequentially as in other examples. The UMZI may comprise one or more phase shifters on at least one arm to change the wavelengths output from each UMZI output port. In this example, the light switch is replaced by a wavelength splitter that may be adapted to enable different wavelengths to be incident upon the different eyes at the same time. For example, after the first set of wavelengths have been input into the first eye E1 (and the second set into the second eye E2), the UMZI may switch the wavelength sets around to be output upon the respective other output port so that eye E1 receives the second set of wavelengths whilst eye E2 received the first set of wavelengths. Upon return, the UMZI recombines the two eye spectra into one mode which propagates through the OCT system as usual. In practise, any device or combination of devices that can separate and then recombine different wavelength portions of the incoming light may be used in place of the UMZI such as but not limited to ring resonators, AWGs, or dichroic mirrors. The wavelength separation can separate wavelengths into two adjacent groups but can also separate into any number of groups and/or have the groups not be adjacent to one another (e.g., interleaved).

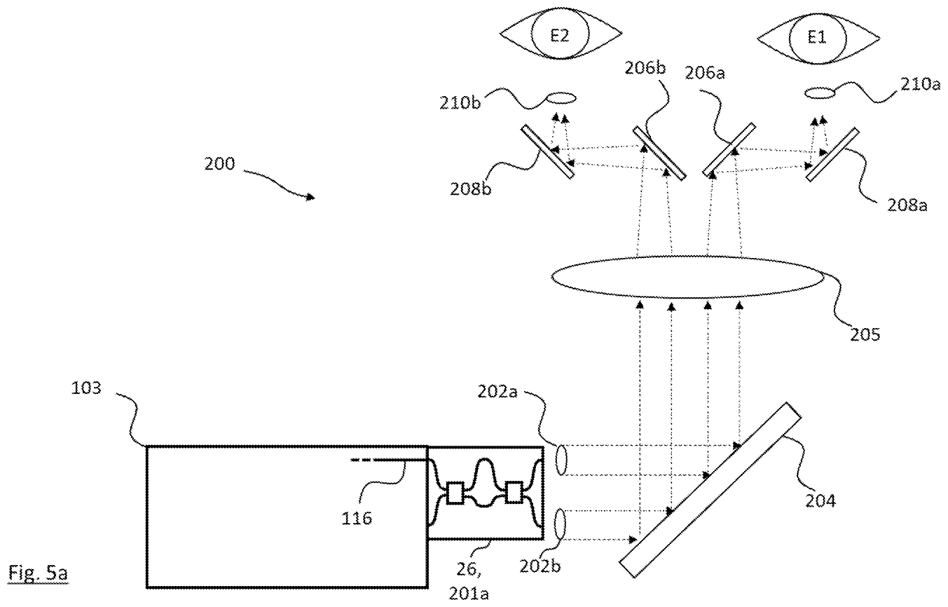

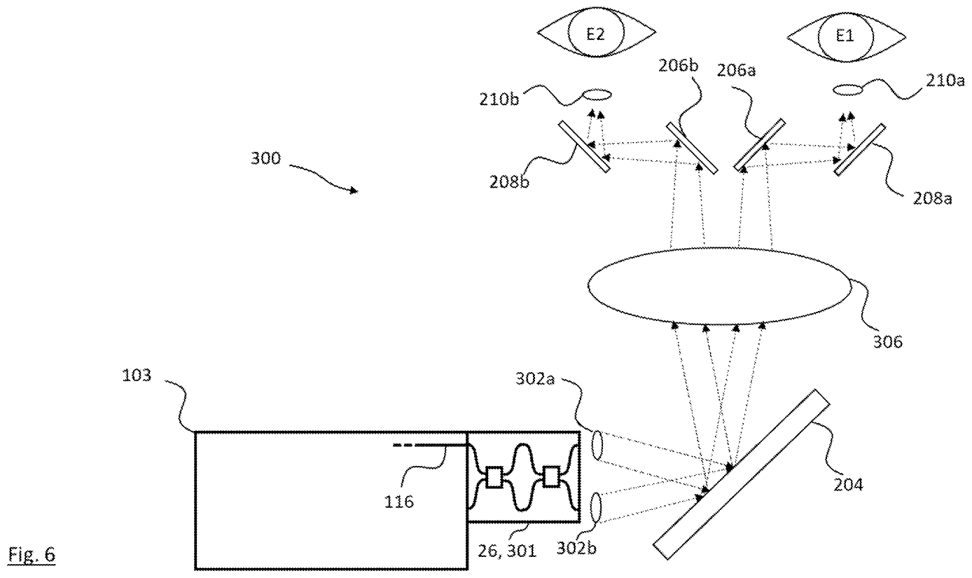

FIG. 6 shows an example 300 using the chip 103 from FIG. 3 having probe output path 116. FIG. 6 is similar to FIG. 5. In this example, either: a) light outputting from chip 301 is angled towards the same, or at least partially overlapping, region of the MEMS 204 (in that the angles of the output waveguides from chip 301 are not parallel to each other but take a convergent path); and/or, b) collimating lenses 302a and 302b each comprises an optical setup (not shown) comprising bulk optical components that direct the separate output light paths from chip 301 towards the same, or at least partially overlapping, region of the MEMS 204. Because light from each different probe path (for E1, E2) is incident upon the MEMs 204 at a different angle, it is reflected at a different set of scanning directions (each set being the scanning X/Y light propagation directions). A strongly convergent lens 306 is used to divert light towards a similar back-end set up as FIG. 5 using mirrors and further lenses.

FIG. 6a is an example similar to FIG. 6 except that the chip 301 is replaced by chip 301a. The chip 301a is similar to chip 301 except that after the MZI, the output waveguides are brought into close proximity on the chip 301a so that they both output light into the same collimating lens 302. Each MZI output port waveguide (of chip 301a) has a lengthwise direction that subtends an angle at the chip edge (about the major plane of the chip) that is different to the angle of the other MZI output waveguide. As such each said waveguide outputs light at a different angle from the chip edge, which in turn is collimated by common lens 302 but is incident upon a different portion of MEMS 204. This configuration has one less lens that FIG. 6. Furthermore, angled waveguide outputs on the chip 301a reduce reflections from the chip edge, particularly back into the waveguide mode.

FIG. 7 shows an example 400 using the chip 103 from FIG. 3 having probe output path 116. No chip 201, 301 is used here, but a single probe path light exiting from chip 103 is collimated by lens 402 to be incident upon MEMS 204 to be focused by lens 205. The apparatus 400 of FIG. 7 is similar to that of FIG. 2 except that the separate mirrors 206a/b are replaced by a common moveable mirror 406 which acts as the switch 26. When needing to test eye E1, the mirror 406 rotates about a pivot to direct light to mirror 208a. When needing to test eye E2, the mirror 406 rotates about a pivot to direct light to mirror 208b. The mirror 406 is moved by an actuator (not shown) upon receiving a control signal.

FIG. 8 shows an example 500 using the chip 103 from FIG. 3 having probe output path 116. A switching chip 501 is used similar to that of FIG. 5. Each spatially separate probe-path output from chip 501 is optically coupled into a different and separate respective piezo electric actuator 502a, 502b that acts, upon receiving control signals, to provide the x-y lateral scanning. Collimating optics (not shown) may be used to focus or collimate light into the piezoelectric actuators 502a/b or optical fibres may be used to guide light from chip 501 to piezoelectric actuators 502a/b. Actuator 502a is for scanning eye E2 wherein the output of actuator 502a is incident upon a set of one or more collimating/focusing lenses 504a, 506a of a lens system that delivers probe light into Eye E2. Other optical components such as mirrors to re-direct light (not shown) may be used. Actuator 502b is for scanning eye E1 wherein the output of actuator 502b is incident upon a set of one or more collimating/focusing lenses 504b, 506b of a lens system that delivers probe light into Eye E1. Other optical components such as mirrors to re-direct light (not shown) may be used.

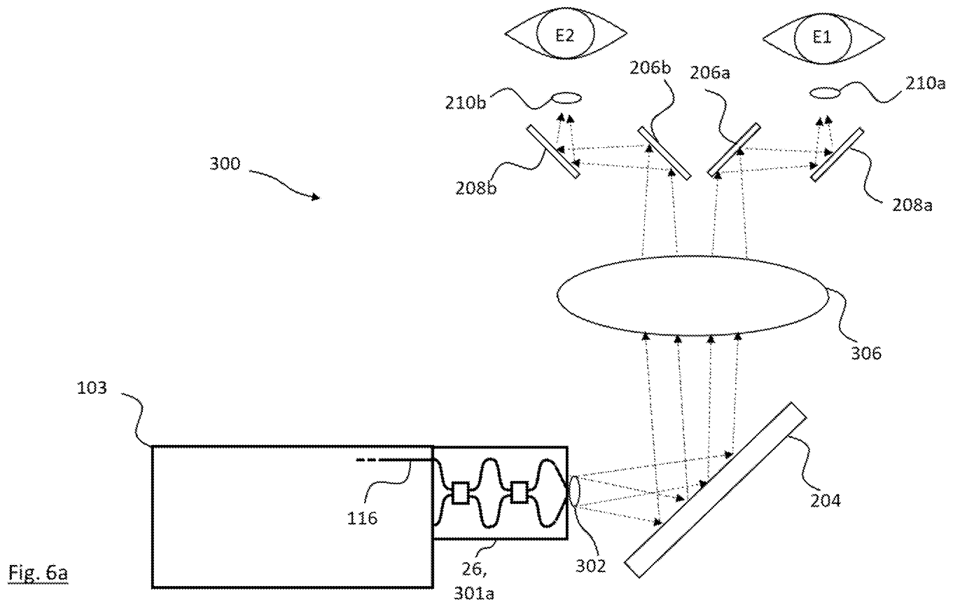

FIG. 9 shows an example 600 using the chip 103 from FIG. 3 having probe output path 116.

No chip 201, 301, 501, is used here, but a single probe path light exiting from chip 103 is input (optionally via a collimating lens or optionally via an optical fibre connection) to be input into an arrangement 601 (for example a hybrid integration package) that acts as a switch 26 to divert light into different probe paths for eyes E1, E2. The arrangement 601 in FIG. 9 comprises a piezo-electric actuator 601a and at least one collimating lens 601b for controllably diverting the light towards either: a) MEMS 602a for X-Y scanning the Eye E2, wherein light reflected from MEMS 602a is routed through a system of lenses 604a, 606a (and optionally other optical components) for inputting probe light into eye E2; b) MEMS 602b for X-Y scanning the Eye E1, wherein light reflected from MEMS 602b is routed through a system of lenses 604b, 606b (and optionally other optical components) for inputting probe light into eye E1. The arrangement 601 may pivot about an axis.

When needing to test eye E1, the piezo-electric actuator 601a rotates the arrangement 601 about a pivot to direct light to MEMS 602b. When needing to test eye E2, the piezo-electric actuator 601a rotates the arrangement 601 about a pivot to direct light to MEMS 602a. The piezo-electric actuator 601a rotates the arrangement 601 about a pivot upon receiving a control signal.

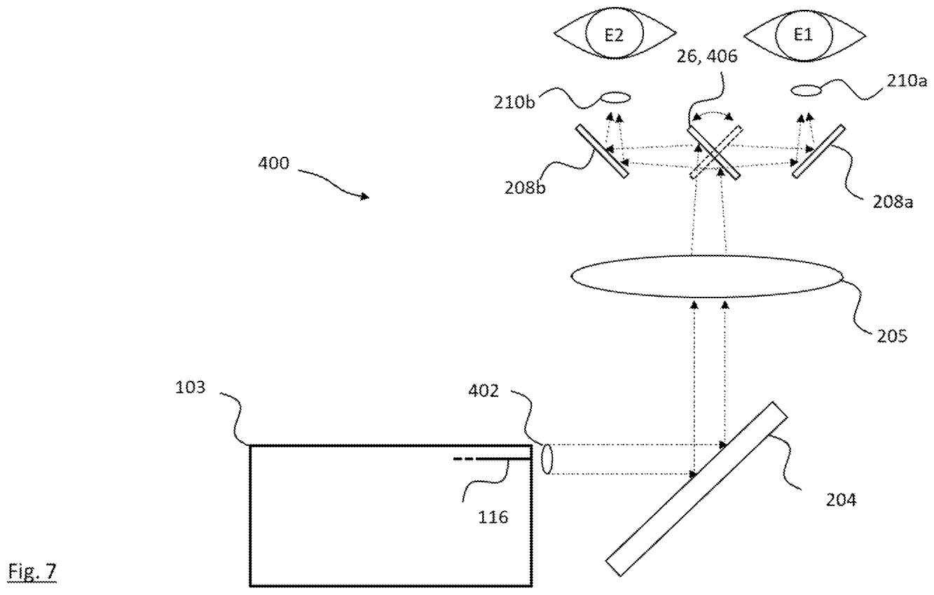

FIG. 10 shows an example 700 similar to FIG. 5 except that no chip 201 is used here but a single probe path exiting from chip 103 is used to input light into collimating lens 702 for focusing onto MEMS 704. Here the MEMS 704 is located upon a translation stage (not shown) and configured to move 701 along at least one axis to output light into different optical set-ups for each eye E1, E2. In this example, the light is input onto different portions of lens 205, for each different translated MEMS position. In a first position about the translation stage the MEMS 704 directs light onto a first region of lens 205 for inputting to mirror 206b for eye E2. In a second position about the translation stage the MEMS 704 directs light onto a second (different) region of lens 205 for inputting to mirror 206a for eye E1.

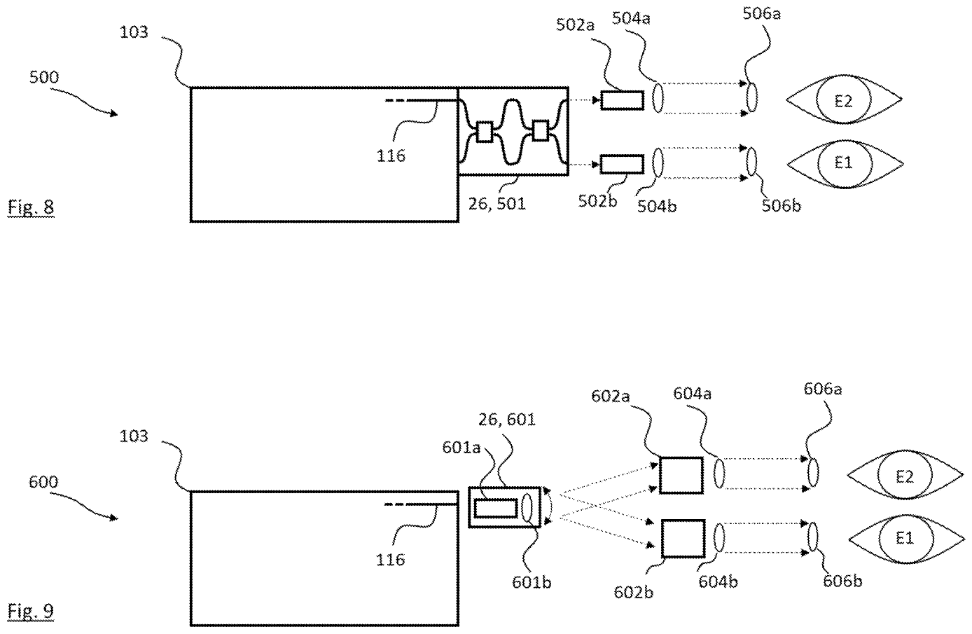

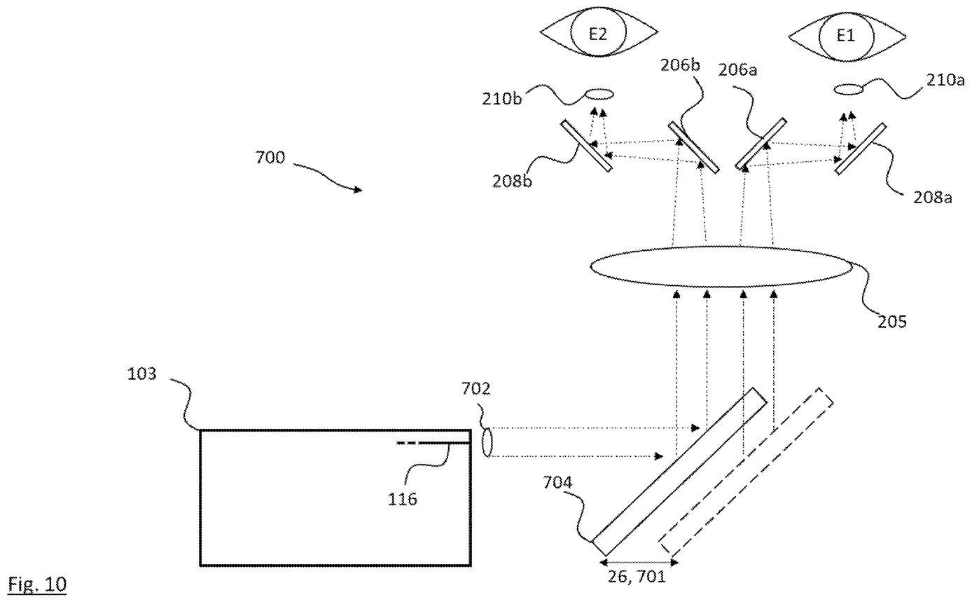

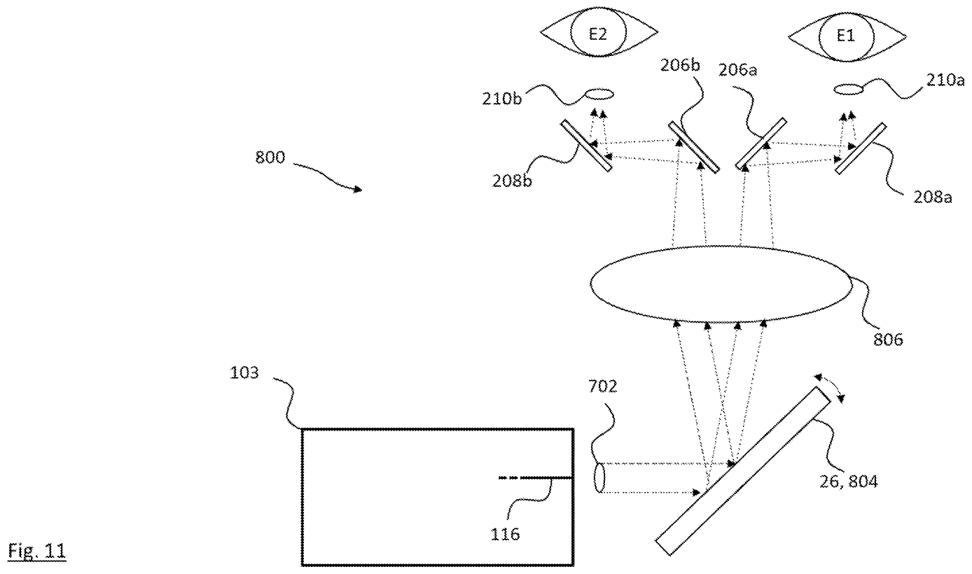

FIG. 11 shows an example 800 similar to FIG. 10 except that light output via collimating lens 702 is incident upon a MEMS 804 that is controllably pivotable (i.e. acting as a switch 26) to: a) divert x-y scanning probe light into a first region of lens 806 for inputting probe light onto mirror 206b for eye E2; b) divert x-y scanning probe light into a second region of lens 806 for inputting probe light onto mirror 206a for eye E1. The MEMS 804, as in other examples outputs a range of light angles corresponding to the desired X-Y scan position of the eye E1, E2; thus, the MEMS 804 is configured via: i) scanning control signals; and, ii) eye-switching control signals, to output a first range of light directions for eye E1 and a second, different, range of light directions for eye E2.

FIG. 12 shows an example 900 similar to FIG. 10 except that light output via collimating lens 702 is either incident upon a first MEMS 904a for directing light towards eye E2 or a second, different, MEMS 904b for directing light towards eye E1. The switching 26 mechanism here is that the light output from lens 702 is nominally incident upon MEMS 904a, however the MEMS 904a may be moveable to not receive the said light and thus allow the light to be incident upon MEMS 904b. The MEMS 904a may be moveable in different ways include being translated or pivoted to not intercept the light from lens 702.

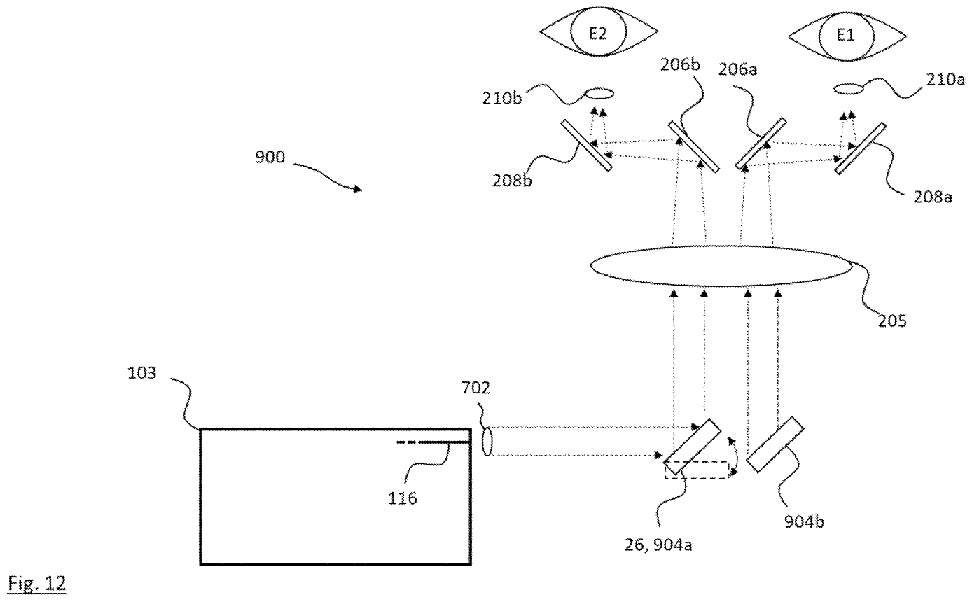

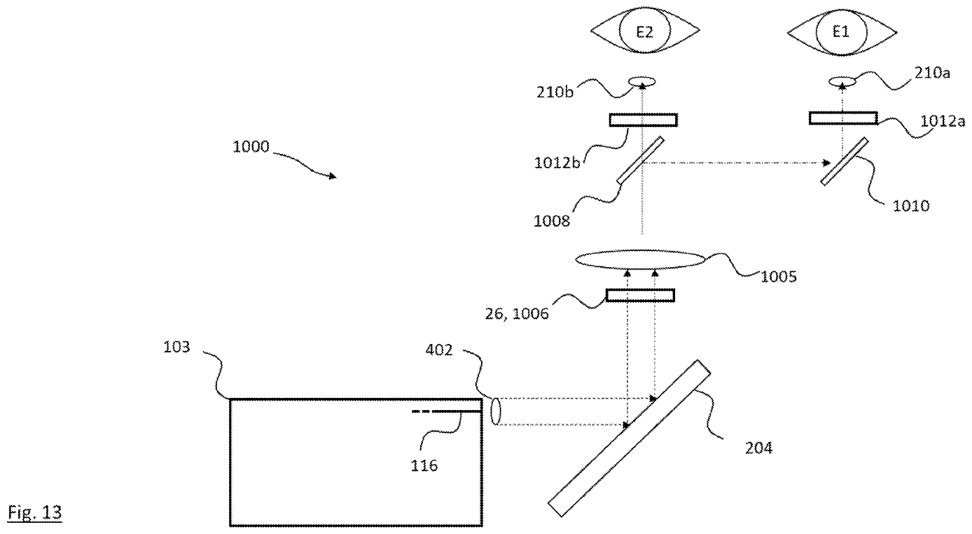

FIG. 13 shows an example 1000 similar to FIG. 7 wherein probe light directed towards the eyes E1/E2 and output from MEMS 204 is incident upon a polarisation rotator 1006 which outputs light into lens 1005 (acting in a similar way as FIG. 7's lens 205). A polarising beam splitter (PBS) 1008 directs (reflects in FIG. 13) one polarisation (for example horizontally polarised) towards mirror 1010 for reflecting probe light towards eye E1, optionally through polarisation compensating element 1012a, and lens 210a. PBS 1008 transmits the orthogonal polarisation through towards eye E2 optionally through polarisation compensating element 1012b, and lens 210b. The polarisation rotator 1006 may be a half wave plate. The polarisation rotator 1006 is controllable to switch 26 light between probe light in a first polarisation that gets routed towards eye E1 and a second polarisation that gets routed towards eye E2. Other polarisation components (not shown) may be used including a polariser in between lens 402 and rotator 1006 that outputs a single light polarisation.

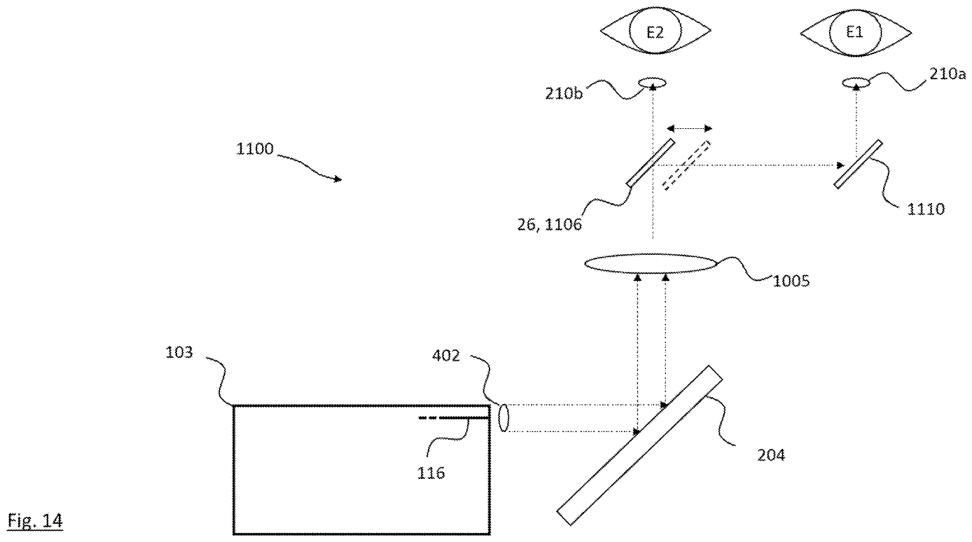

FIG. 14 shows an example 1100 similar to FIG. 13 wherein probe light directed towards the eyes E1/E2 and output from MEMS 204. FIG. 14 does not utilise: the polarisation rotator 1006, nor the polarisation compensators 1012a/b. The PBS of FIG. 13 is replaced by a mirror 1106 that is moveable, translationally, about at least one axis, for example by having the mirror 1106 being mounted on a translation stage (not shown). The switching 26 is facilitated by the mirror being moveable between: a first position wherein the mirror 1106 intercepts and receives the light output from the (optional) lens 1005 and directs it towards a further mirror 1110 for directing the light to eye E1 via lens 210a; b) a second position wherein the mirror does not receive the light output from the MEMS 204 toward the eyes E1, E2, wherein the light propagates through to the lens 210b for input to eye E2.

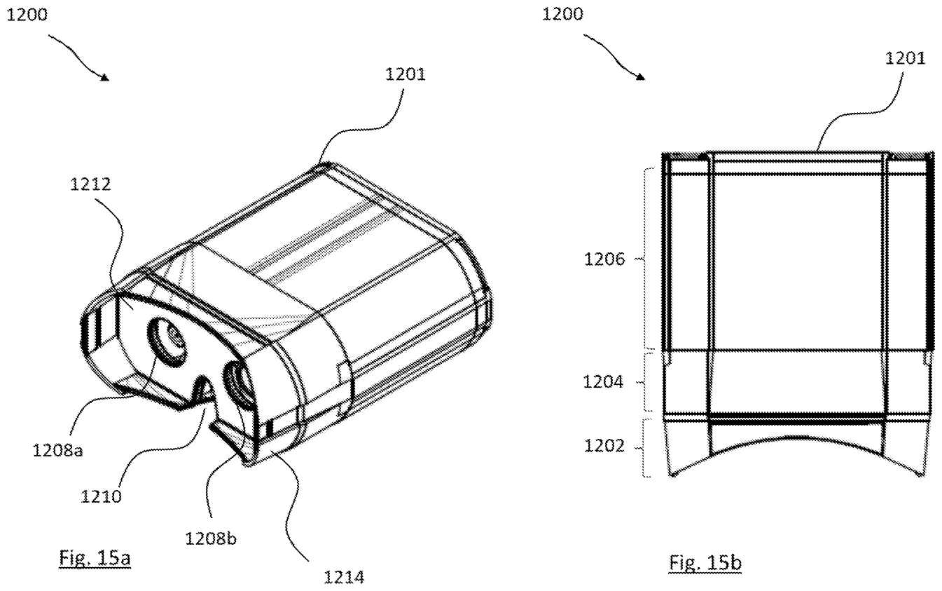

FIGS. 15a and 15b show an example of a OCT system 1200 that is portable and optionally head mountable. The figures show the outer housing 1201 of the OCT system that may optionally accommodate all or a portion of the OCT apparatus 2 from FIG. 1 or 2 or from other examples herein. The apparatus 2 may comprise other features described herein including, but not limited to: the light source 6; any detector arrangements; control electronics; computational components for receiving electrical signals from a detector and processing those signals and/or for determining drive signals for controlling any of the components of the OCT system; communications components for transmitting and/or receiving data to or from a third party; electrical power sources for supplying electrical power to any of the component requiring such power, probe path components for delivering and collecting light from the eyes E1, E2.

The housing 1201 may have different regions. The regions may be separable, hence the housing may be modular. The separable sections may be attached using any attaching means or mechanisms inside or outside the housing. The OCT apparatus accommodated by the housing 1201 may also be separable into sets of components wherein a set of components may form part of one or more regions of the housing 1201. Having a modular system allows for simple assembly and repair and improved lifetime. In addition, it can create a means for optical and vibrational isolation and thermal insulation. Each region may be for a different function and/or for housing different components. FIG. 15b shows an example of three regions of the housing 1201, namely a front region 1202, middle region 1204 and rear region 1206. The middle region 1204 and rear region 1206 may house components of the OCT apparatus 2. The housing 1201, in particular each of the middle 1204 and rear 1206 regions may comprise a plurality of walls that at least partially define a housing chamber that accommodates the apparatus 2. The walls may comprise outer walls having at least one surface adjacent to the outside environment and inner walls that are enclosed by the housing 1201. The housing chamber may be substantially surrounded on all sides by the outer walls and may be sealed from the outside environment by the said outer walls, for example being hermetically sealed. The housing chamber may accommodate the inner walls and thus may define the inner housing space that accommodates the OCT apparatus. One or more inner walls may be used for sub-dividing the housing chamber into a plurality of compartments. At least one inner wall may define a boundary wall between the middle region 1204 and rear region 1206. Any of the inner walls may be separable to the outer walls such that the inner wall may be removable from the housing chamber. Alternatively, any of the inner walls may be integral with one or the outer walls that define one of the middle 1204 or rear 1206 regions.

In use, when a user is using the OCT apparatus in the housing 1201 to perform an OCT measurement:
a) the front region 1202 is located between the middle region 1204 and the user's face; the front region 1202 may engage the user's face by coming into contact.
b) the middle region 1204 is located between the rear region 1206 and the front region 1202 and user's face.

Any of the walls of the housing 1201 may be formed of any suitable material. Preferably the material is opaque apart from, at least, portions where light is required to enter or exit the chamber. The wall material is preferably substantially rigid. The wall material is preferably non-porous. An example of a wall material may be a metal or a plastic.

The housing regions 1202, 1204, 1206 (and hence housing 1201) have one or more widths that, when in use, are the dimensions that extend substantially parallel to the users face from first eye to second eye. The depth of the regions 1202, 1204, 1206 is the dimension, when in use, that extends substantially parallel to the users face and perpendicular to the width (hence running substantially parallel to the direction from the top of the user's head to their chin. The length of the regions 1202, 1204, 1206 is the dimension perpendicular to the width and depth.

The middle region 1204 may comprise substantially the same width and depth as the rear region 1206. The rear region 1206 may comprise a length less than, greater than or substantially the same as, the middle region. The front region 1202 may have a first width at an end proximal to the middle region 1204 and a second width at an end proximal to the user's face, when in use. The first width may be greater than the second width. The end of the front region 1202 for engaging with the user's face may be open in that the said front region end is a large open aperture defined by the face-proximal edges of one or more outer walls 1214 running and defining the length of the front region 1202.

The front region 1202 is proximal to the user's face (not shown) in use. The front region 1202 may comprise two portions for allowing probe light to travel to/from the user's eyes from/to the middle region 1204. These portions may be located within a wall 1212 separating the front region 1202 from the middle region 1204. The two portions in this example comprise through holes through the wall 1212. The through holes may allow air through to the middle region 1204 or may comprise material forming transparent windows wherein the transparent material may extend laterally across the through holes to seal the said holes 1208a/b. Any of the windows may comprise a lens of the apparatus 2.

The housing 1201 may comprise a nose-notch 1210 for fitting around the user's nose when in use. Thus, at least the front region 1202 is preferably shaped to engage with the users face and include the nose-notch 1210. The middle region 1204 may also be shaped to jointly form the nose notch with the front region 1202.

This example may have fewer or more regions than shown and described, for example, the housing 1201 may not include a front region wherein the wall 1212 proximal to the user's face (in use) is the part of the housing that contacts the user's face.

The housing 1201 may be referred to as a portable apparatus for accommodating an ocular Optical Coherence Tomography, OCT, system. The OCT system may comprise the apparatus 2 described elsewhere herein or other OCT systems. When accommodating the apparatus 2 or other OCT system in the housing 1201, the OCT system may be regarded as comprising: at least two sets of components. A set of one or more optical components may be at least for directing light from a light source to at least one eye of a user when wearing (or otherwise using) the portable apparatus. A further set may comprise one or more further components wherein at least one of the further components is an electrical component. The set of optical components may be referred to as the ‘first set’ whilst the further set may be referred to as the ‘second set’. Other sets of components may be included, for example the OCT apparatus contained within the housing 1201 may comprise two or more, or three or more sets of components. The electrical component may be for controlling at least one of the optical components of the set of optical components, and/or, another of the further components. For example, an electrical component of the second set may be control electronics for controlling the movement of a mirror in the first set.

As described above, the portable apparatus may comprise a plurality of compartments. The compartments may comprise a first compartment for accommodating the set of one or more optical components. The compartments may comprise a second compartment for accommodating the set of one or more further components.

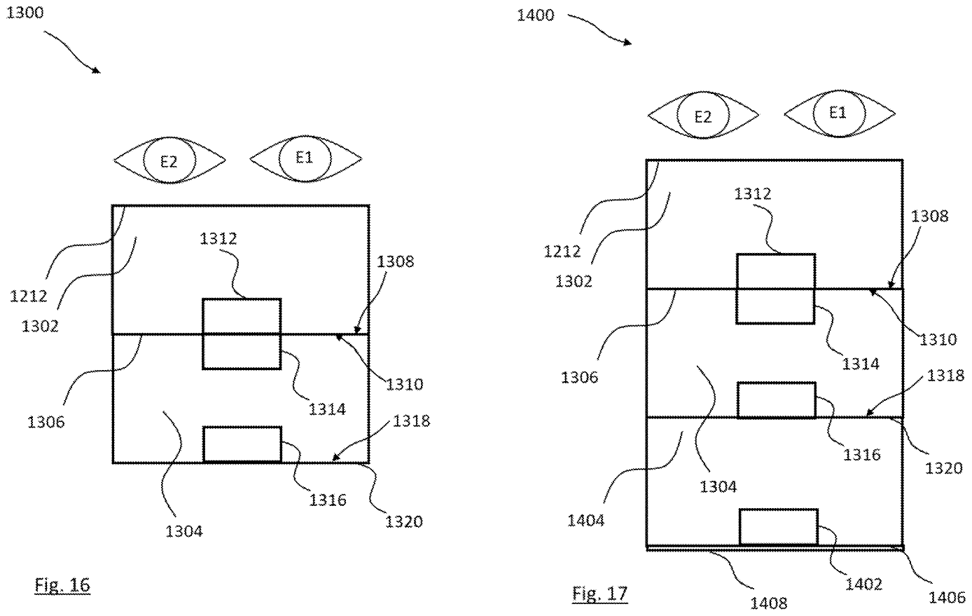

FIG. 16 shows a first example of the portable apparatus 1300 that is similar to FIGS. 15a and 15b except that no front region 1202 is used and the wall nearest the user's face is the same wall 1212 as in FIG. 15a. FIG. 16 shows the cross section along a length-width plane of the apparatus 1300. The portable apparatus 1300 comprises the first compartment 1302 and the second compartment 1304. In some examples, the first compartment 1302 may be similar to the middle region 1204 whilst the second compartment 1304 may be similar to the rear region 1206. An inner wall 1306 is positioned between the first compartment 1302 and second compartment 1304 and extends substantially across the width and depth of portable apparatus 1302 so as to provide thermal insulation and optical isolation between the first compartment 1302 and the second compartment 1304.

The inner wall 1306, when, mounted in the housing, may optionally create a sealing between the first compartment 1302 and second compartment 1304. The sealing may be airtight and/or watertight. Optionally, this may be facilitated by the inner wall 1306 or be an integral part of the middle region 1204 or the rear region 1206. Where the inner wall 1306 is separable, then optionally, one or more sealing members, such as adhesive or rubber seals may be used to seal and fix the inner wall 1306 in place with respect to the rest of the housing. In other examples the inner wall 1306 may not fully seal the first compartment 1302 from the second compartment 1304 but may provide one or a plurality of small through holes for the passing of optical connecting or electronic connecting elements (such as optical fibres or electrical wires) and/or one or a plurality of electrical vias between the two compartments. The wall may cover over 90% of the boundary between the first 1302 and second 1304 compartments; preferably over 95%, more preferably over 99%. Preferably the material of the inner wall 1306 (and optionally other walls) is an electrical insulator. Preferably the material of the inner wall 1306 (and optionally other walls) is thermally insulating. Preferably the material of the inner wall 1306 (and optionally other walls) is optically isolating, although it may also be substantially transparent or provide one or a plurality of small transparent windows for the free-space transmission of light. Preferably, the material of the inner wall 1306 (and optionally other walls) is vibrationally isolating. Preferably the material may be a plastic that is hard and rigid. The material may be combined using any technique (for example, lamination) with a second, flexible material to isolate one compartment from vibrations that may exist in another compartment. The wall 1306 may be constructed from a single piece of material or may be constructed from multiple pieces of the same material separated by a passive isolation system, such as but not limited to pneumatic isolators or mechanical springs, to isolate one compartment from vibrations that may exist in another compartment. The wall 1306 preferably has a thickness over the majority of the plane separating the first/second 1302/1304 compartments greater than 5 mm, or any of: 6 mm or greater, 7 mm or greater, 8 mm or greater.

The operation of optical components can sometimes be undesirably affected by heat from other components. By having the optical components 1312 in one compartment 1302 and heat-generating electrical components 1316, 1314 in a separate, second compartment 1304 thermally insulated from the first compartment, the optical components 1312 suffer less from unwanted heat.

The operation of optical components can sometimes be undesirably affected by vibrations from other components. By having the optical components 1312 in one compartment 1302 and vibration-generating components 1316, 1314 in a separate, second compartment 1304 vibrationally insulated from the first compartment, the optical components 1312 suffer less from unwanted vibrations. An example of a component that may generate unwanted vibrations includes but is not limited to a mechanical fan that may be used to maintain temperature stability within the second compartment 1304.

The OCT device contains light emitting and light measuring/detecting components, as well as an interface with a sample that may be light sensitive (an eye). Compartmentalising the device with an opaque wall between compartments allows for optical isolation between compartments and/or between the device and the outside world. This improves the performance of the device, for example by removing noise caused by any stray ambient light incident on the detection system, and can also improve its safety.

The lifetime of components within such a device is often limited by interactions with the atmosphere or environment. For example, moisture in the air often leads to failure of electronic components or interfaces. Compartmentalising the device, particularly with barriers and/or hermetic sealing, will decrease the shared environment that the components sit in and therefore is likely to extend the lifetime of the device as a whole.

Any device involved in medical practice will need to be regulated and/or ISO compliant and quality managed. A modular device, with modules which are interfaced at particular points, provides an easier route with which to monitor development, operation, functionality, and performance. A modular device additionally allows for easier iteration or changes of device design as changes can be flagged for the particular modules that the change is applied to, such that review or paperwork required for the changes can be applied to the changed module(s) and not to the other unchanged module(s).

The inner wall 1306 may be substantially planar. The inner wall 1306 may be a plate comprising a first major face 1308 (facing in a first direction) and a second major face 1310 (facing in a second direction opposite the first direction). The inner wall 1306 may be removable, hence separable, from the outer housing walls of the portable apparatus 1300. In use, the first major face 1308 faces the user's face. Upon the first major face 1308 is mounted at least one of, preferably all of, the first set of one or more optical components 1312. Upon the second major face 1310 is mounted at least one of, optionally all of, the second set of components 1314. A further, third, set of one or more components 1316 is shown in this example 1300. The third set 1316 is optionally mounted upon an inner surface 1318 of an end wall 1320 of the second compartment 1304. The end wall 1320 being at the opposing end of the second compartment 1304 to the inner wall 1306 and forming one of the outer walls of the housing.

The optical components 1312 may comprise any one or more of, but not limited to: a light source 8; interferometer splitter 9; interferometer combiner 22; optical elements defining the reference arm, optical elements defining at least a portion of the probe arm; any of the x, y or z scanning optics; any optical elements guiding or otherwise directing light from one optical component to another; any components for moving optical components such as translation stages or actuators. In this example, the optical components 1312 comprise the chip 103 of FIG. 5 and other optical components comprising the probe arm of FIG. 5. The lenses 210a/b may instead be mounted within or on the outer wall 1212 as described above. Equivalently, any of the examples of probe path optics in FIGS. 6-14 may also form the set of optical components 1312. The second set 1314 comprising control electronics for driving any of the components in the first set 1312 may comprise control electronics for driving any one or more of, but not limited to: any of the actuators for controlling the position of optical components; any of the switching components on chip such as thermo-optic heaters; the light source; the detection system.

The third set 1316 may comprise any one or more of, but not limited to: a computer processor; a communications transceiver; a power supply unit for supplying electrical power to any of the electrically driven components of the apparatus such as the processor and control electronics; any communication hardware such as a WiFi or mobile transceiver, required for sending or receiving information from the cloud, base unit, or other device.

The position, about the housing, of at least the second set of components 1314, and optionally, where applicable, any third 1316 or further set of components, may be changed from that shown in FIG. 16 and other figures herein. In some examples the portable apparatus 1300 may be head mountable such that the apparatus 1300 comprises one or more features to mount the apparatus 1300 onto the user's head such that the user does not have to physically hold the apparatus with their hands, nor does a further person need to hold it for them, nor is any form of mount, such as but not limited to a tripod, required. The features may therefore be securing features such as one or more straps, or a helmet, that hold the apparatus 1300 in place whilst the OCT measurement is being performed. Being held in place meaning to maintain a relatively stable position with respect to the person's face such that the probe light does not need re-aligning or re-focusing for a single depth scan, b-scan, or en-face. For such a head mountable portable OCT apparatus having electrical components 1314 situated in the middle or near the front (near the persons face) of the apparatus 1300 may bias more of the weight of the entire apparatus nearer the persons face. This has the advantage of having less weight at the far extremity of the apparatus 1300 near the rear wall 1320. More weight at or near the rear wall 1320 would increase the chance of the apparatus either falling off the user's head, adding undue strain to their neck, creating undue pressure on their nose, or otherwise moving and requiring the OCT measurement to be re-performed. Weight distribution towards the users face also increases comfort. If the apparatus 1300 is a head mountable version, then it is a ‘wearable’, as such. When head mountable, other electronic components such as third set 1316 may instead be mounted upon the inner wall 1306 or disposed proximal to the first compartment 1302. The third set 1316 may be affixed to the inner surface of an outer wall of the second compartment. Preferably the inner wall 1306 is closer to the user-facing outer wall of the first compartment 1212 (or otherwise nearer the user's face, when in use) than the rear wall 1320 of the second compartment 1304 (or otherwise the rear wall of the entire housing). Preferably the first compartment 1302 has a shorter length than the second compartment (or a shorter length than the sum of the lengths of the other compartments positioned outwardly away from the users face from the inner wall 1306). In this example of a head mountable apparatus, preferably a larger proportion of the weight of the apparatus 2 components is disposed closer to the housing outer wall 1212 (facing the users face, when in use) than the rearmost wall 1320 of the housing. Additionally, or alternatively at least one set of components, may be located upon a further inner wall (not shown in FIG. 16) located in the user-proximal half of the length of the housing; wherein such components may be located in the second 1304 or an optional further compartment.

In another example, the portable apparatus 1300 may be a handheld apparatus, in that the user is required to hold the portable apparatus 1300 during an OCT measurement or support the portable apparatus 1300 on a mount, including but not limited to a tripod, at a point that may be central in the length and width of the portable apparatus 1300 during an OCT measurement. The apparatus 1300 may be a binocular apparatus. In this example it is advantageous to balance the weight of the entire portable apparatus 1300 (which includes OCT apparatus 2) throughout the length of the body of the portable apparatus 1300 because people find it easier to hold and maintain an object in a steady position when weight is distributed more evenly, or, if a mount is required, then the apparatus 1300 will more easily remain level (and/or more stable) relative to the user if it is balanced around the mounting position.

Preferably: a) at least 30% of the weight of the entire portable apparatus 1300 is disposed in the user-proximal half of the apparatus 1300 (‘half’ being divided along the length of the apparatus 1300); at least 30% of the weight of the entire portable apparatus 1300 is disposed in the user-distal half of the apparatus 1300. More preferably: a) at least 40% of the weight of the entire portable apparatus 1300 is disposed in the user-proximal half of the apparatus 1300 (‘half’ being divided along the length of the apparatus 1300); at least 40% of the weight of the entire portable apparatus 1300 is disposed in the user-distal half of the apparatus 1300.

For this example of use, at least one set of components, (for example the second set 1314 and/or third set 1316) may be mounted upon the rear wall 1320 of the second compartment and/or the rearmost wall of a compartment furthest from the users face during use. Additionally, or alternatively at least one set of components, (for example the second set 1314 and/or third set 1316) may be mounted upon any of: a) the rear wall 1320; b) a portion of an inner surface of an outer wall, (proximal to the rearmost wall), extending along the length of the housing; c) a further inner wall (not shown in FIG. 16) located in the user-distal half of the length of the housing.

The components that are preferably located upon the rearmost wall may include any one or more of, but not limited to: a power supply and/or battery, a computer processor, a communications transceiver. It is to be understood that the first and second (and possibly other) compartments may comprise optical or electrical components.

FIG. 17 shows a further example of a portable apparatus 1400 that is similar to FIG. 16 with like references indicating like components. A further, third, compartment 1404 is located adjacent to the second compartment 1304 and separated by the wall 1320. Wall 1320, at the user-distal end of the length of the second compartment 1304, is an inner wall in this example and may be referred to as a ‘second’ inner wall. The inner wall separating (hence dividing) the first 1302 and second 1304 compartments, may be referred to as the first wall. Any options for the first wall 1306 described herein may apply to the second wall, including being optionally separable from the housing. The third compartment 1404 comprises a user-distal rear wall 1406 upon which is mounted: a) a fourth set of components 1402 on an inner surface; and b) an induction coil 1408 for wireless charging on the outer surface. This induction coil 1408 may be applied in a similar position to the rearmost wall (or another outer wall) of the housing. In this example the third set of components 1316 comprises a processor and optionally memory and or other components; whilst the fourth set of components 1402 comprises a power supply unit electrically connected to receive power from the induction coil 1408. In some examples the induction coil 1408 may be an induction unit that forms the rear wall of a compartment.

FIG. 18 shows a further example of a portable apparatus 1500 that is similar to FIG. 17 with like references indicating like components. In this example the third set of components 1316 is mounted upon the major surface of the second inner wall 1320, that faces away from the user (when in use).

In any of the examples above a heat sink and/or fan may be included upon the housing, preferably mounted upon the rearmost wall. In such examples, it is preferable that the power supply unit and optionally, other heat generating apparatus, are located upon the wall that also mounts the heat sink.

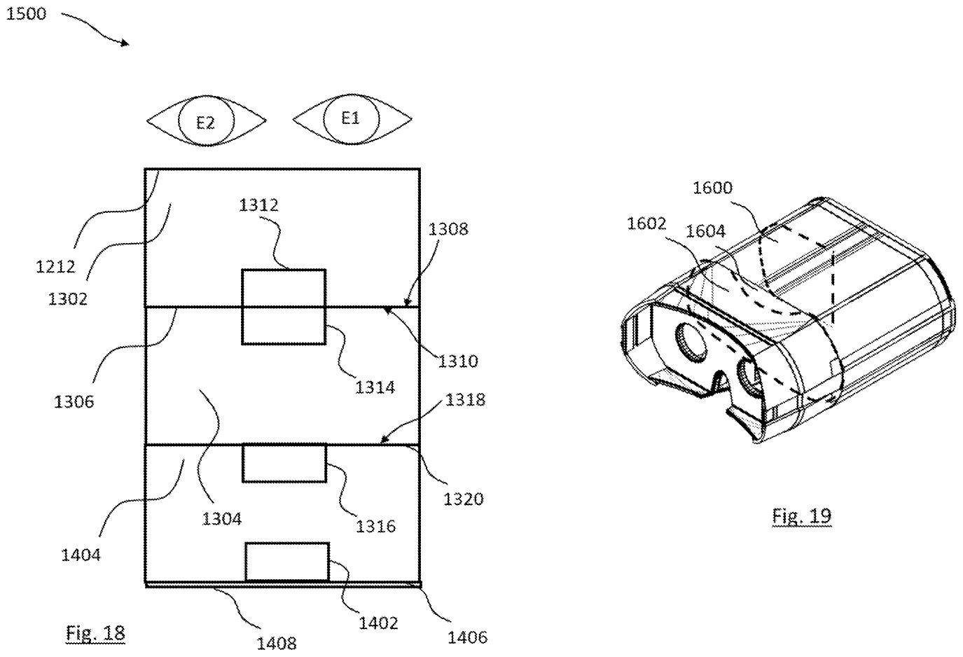

Some internal walls may be included that do not extend across the width of the housing as shown in FIG. 19. Such walls may be platforms 1600 to locate components upon but not subdivide compartments. In FIG. 19 an example of a first inner wall 1602 is shown with a gap 1604 allowing the formation of a substantial through-hole between the first and second compartments 1302, 1304. In this example, the portable apparatus may be assembled by securing the inner wall 1602 in place, making any optical/electrical cable/wire connections required through the gap and then filling the gap 1604 with a filling material (such as a cement or adhesive) to fill the gap and seal the compartments. The same or a similar sealing process may be done for other examples herein with through holes between inner walls separating adjacent compartments.

Walls, be them internal or outer, may have inbuilt features for attachment to components of the OCT apparatus 2 and/or forming part of a component of the OCT apparatus 2, for example being an integrally formed (e.g., moulded) feature. These features are preferably on inner facing surfaces of any walls. Such features may form protrusions and/or depressions upon the planar surface of a wall.

Components of the apparatus 2 such as in the first set, second set etc, may be mounted to features of the housing, such as a wall, in a variety of ways including, but not limited to: any fixing means generally; being secured using adhesive; being friction fitted; being attached via brackets; being attached via one or more screws, being snap fit, being integrally form with a wall, being coated onto a portion of a feature of a wall.

It is to be understood that the examples in FIGS. 16-18 may be adapted with components, configurations and other features as presented in other examples herein. The adaption may be by addition or replacement. Existing features of the examples in FIG. 16-18, may be removed.

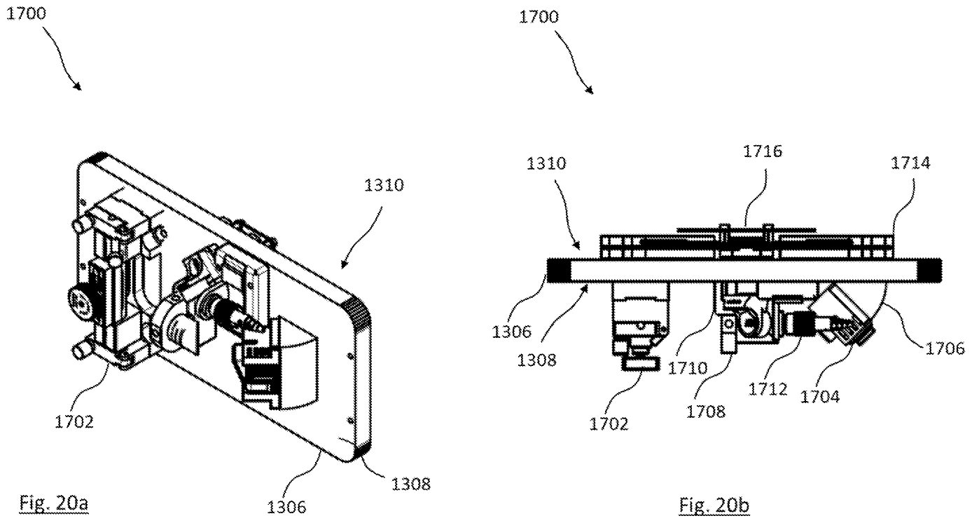

FIG. 20a shows a perspective view of an example 1700 of a first wall 1306 for dividing the first 1302 and second 1304 compartments. FIG. 20b is a side on view of the same example 1700. The wall 1306 is formed of a hard rigid plastic and comprises moulded features to support at least some of the mounted components. In this example 1700, the surface 1308 of the inner wall 1306 separating the first and second compartments 1302, 1304, has the following components mounted upon it: a) a polarisation controller 1702; b) a MEMS scanning mirror 1704 mounted on a raised integral portion 1706 of the wall 1306; c) collimating mirror 1708 mounted on a further raised integral portion 1710 of the wall 1306; d) non-collimating fibre coupler 1712; e) a wall-integrated support 1714 to which optical fibres (for example for use in connecting the probe path output from the chip 103, or for connecting to a reference arm of the OCT interferometer) are fixed using any method of physical attachment including, but not limited to, adhesive, sealant, or a bracket, in order to provide strain relief; f) a photonic chip 1716, similar or the same as chip 103 in previous figures herein, wherein the chip 1716 may be wire-bonded to a PCB board. It should be understood that this example may be adapted, for example by removing the optical fibre-based components including the supports 1714.

The polarisation controller 1702 may be used for correcting for polarisation rotation of light in the optical fibres. As described above, the apparatus may use free space propagation for the probe light, hence polarisation control may not be required in some examples. A human eye has birefringence and therefore in some examples of the apparatus 2, a polarisation controller may be required along the probe and optionally reference path. The location of the polarisation controller may be an integrated-optic component on a chip (for example on chip 103 and/or 201). The above example is merely showing an example of various optical components used for an example of a probe-path for an apparatus 2. The apparatus 2 and portable apparatus, 1300, 1400, 1500 may comprise other features such as but not limited to: user interfaces such as button or displays; features for releasably securing the portable apparatus to a person; communication apparatus such as wired or wireless receivers, transmitters or transceivers for sending/receiving data to/from the portable apparatus. Such data may be data associated with an OCT measurement result or data for controlling the OCT apparatus 2.

The chip 1716 is shown to reside mounted upon the second compartment-facing major planar surface 1310 of the inner wall 1306, however in other examples it may be located on the opposite surface 1308 of the same wall.

There now follows optional details for implementing computer-related aspects of the above apparatus and method. Some portions of the above description present the features including one or more computers comprising a processor and a memory wherein the memory comprises instructions which when executed by the processor give rise to certain steps such as controlling a switching arrangement. These instructions may take the form of algorithms. It should be understood that the process steps, instructions, of the said method/system as described and claimed, may be executed by computer hardware operating under program control, and not mental steps performed by a human. Similarly, all of the types of data described and claimed may be stored in a computer readable storage medium operated by a computer system, and are not simply disembodied abstract ideas.

The method/system/apparatus also relates to an apparatus for performing the operations herein. This apparatus may be specially constructed for the required purposes, or it may comprise a general-purpose computer selectively activated or reconfigured by a computer program stored on a computer readable medium that can be executed by the computer. Such a computer program is stored in a computer readable storage medium, such as, but not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

Any controller(s) referred to above may take any suitable form. For instance, the controller(s) may comprise processing circuitry, including the one or more processors, and the memory devices comprising a single memory unit or a plurality of memory units. The memory devices may store computer program instructions that, when loaded into processing circuitry, control the operation of the route provider and/or route requester. The computer program instructions may provide the logic and routines that enable the apparatus to perform the functionality described above. The computer program instructions may arrive at the apparatus via an electromagnetic carrier signal or be copied from a physical entity such as a computer program product, a non-volatile electronic memory device (e.g., flash memory) or a record medium such as a CD-ROM or DVD. Typically, the processor(s) of the controller(s) may be coupled to both volatile memory and non-volatile memory. The computer program is stored in the non-volatile memory and may be executed by the processor(s) using the volatile memory for temporary storage of data or data and instructions. Examples of volatile memory include RAM, DRAM, SDRAM etc. Examples of non-volatile memory include ROM, PROM, EEPROM, flash memory, optical storage, magnetic storage, etc. The terms ‘memory’, ‘memory medium’ and ‘storage medium’ when used in this specification are intended to relate primarily to memory comprising both non-volatile memory and volatile memory unless the context implies otherwise, although the terms may also cover one or more volatile memories only, one or more non-volatile memories only, or one or more volatile memories and one or more non-volatile memories.

The algorithms and operations presented herein can be executed by any type or brand of computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. In addition, the method/system is not described with reference to any particular programming language. It is appreciated that a variety of programming languages may be used to implement the teachings of the invention as described herein.

1-19. (canceled)

20. A portable apparatus for accommodating an Optical Coherence Tomography (OCT) system,
the portable apparatus comprising:
A) a first end face proximal to a user when using the portable apparatus and comprising at least one aperture for directing light to at least one eye of the user;
B) a plurality of compartments comprising at least:
a) a first compartment for accommodating a set of one or more optical components for directing light from a light source to the at least one eye; and
b) a second compartment for accommodating a set of one or more further components of the OCT system; the second compartment spaced from the first end face by at least the first compartment; and
C) a wall positioned between the first compartment and second compartment and extending substantially across the portable apparatus so as to provide thermal insulation between the first compartment and the second compartment.

21. The portable apparatus as claimed in claim 20 wherein the wall is removable from the portable apparatus.

22. The portable apparatus as claimed in claim 20 wherein the set of one or more optical components is mounted on the wall.

23. The portable apparatus as claimed in claim 20 wherein the set of one or more further components is mounted on the wall.

24. The portable apparatus as claimed in claim 21 wherein the portable apparatus comprises a housing body comprising at least: the first compartment, the second compartment; the wall; the first end face; wherein:
the wall is a first wall disposed inside the housing body;
the set of one or more further components being a second set of components;
the first end face is an outer face of a second wall;
the housing body comprises a third wall at a distal end of the housing body directly away from the user's face when in use; the third wall comprising: i) an outer face facing away from the user's face when in use; and ii); an inner face facing towards the user's face when in use; and
the portable apparatus comprising a third set of components mounted upon the inner face of the third wall.

25. The portable apparatus as claimed in claim 24 further comprising at least one of:
i) a heat sink;
ii) a fan; and/or
iii) an induction charging unit;
integrated with the third wall or mounted on the outer face of the third wall.

26. The portable apparatus as claimed in claim 24 wherein the second wall is removable from the portable apparatus.

27. The portable apparatus as claimed in claim 20 wherein the portable apparatus is head mountable.

28. The portable apparatus as claimed in claim 20 wherein the set of one or more optical components comprises a photonic chip, the portable apparatus comprising the photonic chip.

29. The portable apparatus as claimed in claim 28 wherein the photonic chip is mounted upon the wall.

30. The portable apparatus as claimed in claim 20 wherein the wall comprises a through-hole between the first and second compartments.

31. The portable apparatus as claimed in claim 20 wherein the portable apparatus is a binocular apparatus.

32. The portable apparatus as claimed in claim 20 wherein the wall comprises an opaque material.

33. The portable apparatus as claimed in claim 20 wherein the wall provides optical isolation between the first and second compartments.

34. The portable apparatus as claimed in claim 20 comprising:
the set of one or more optical components; and
the set of one or more further components.

35. The portable apparatus as claimed in claim 34 wherein more of a weight of the portable apparatus is nearer the user's face than an opposing end wall at a distal end of the portable apparatus.

36. The portable apparatus as claimed in claim 20 comprising:
the OCT system configured for measuring a first target sample and a second target sample; the OCT system comprising:
I) an optical arrangement configured to:
i) receive light from a light source;
ii) split the received light to propagate along at least: a reference optical path; and, a probe optical path;
iii) receive light back from the reference optical path;
iv) receive light back from the probe optical path; and
v) interfere the light received back from the reference and probe optical paths; and
II) a light switching arrangement configured to:
receive light from the probe optical path, and
controllably switch the received light between at least:
a) a first optical path for directing light towards the first target sample; and
b) a second optical path for directing light towards the second target sample.

37. The portable apparatus as claimed in claim 36 comprising an integrated optic interferometer.

38. The portable apparatus as claimed in claim 20 comprising:
the OCT system; the OCT system comprising:
(I) the set of one or more optical components; and
(II) the set of one or more further components wherein at least one of the further components is an electrical component for controlling at least one of:
i) the optical components, and/or
ii) another of the further components.
